(19) United States
(12) Patent Application Publication
(10) Pub. No.: US 2017/0210704 A1
(43) Pub. Date: Jul. 27, 2017

(54) DEUTERO-PHENFORMIN DERIVATIVES

(71) Applicant: Enlibrium Inc, Ja Jolla, CA (US)

(72) Inventor: David CAMPBELL, San Diego, CA (US)

(21) Appl. No.: 15/413,251

(22) Filed: Jan. 23, 2017

Related U.S. Application Data
(60) Provisional application No. 62/281,625, filed on Jan. 21, 2016.

(51) Int. Cl. C07C 279/26; C07B 59/00

(52) U.S. Cl. CPC C07C 279/26; C07B 59/001; C07B 2200/05

(57) ABSTRACT

The present disclosure is directed to novel phenformin derivatives, and their pharmaceutically acceptable salts, solvates, or stereoisomers thereof. This disclosure also provides compositions and the use of such compositions in method of treating cancer, diabetes, or polycystic ovarian syndrome.

### CROSS-REFERENCE

This application claims the benefit of U.S. provisional application Ser. No. 62/281,625, filed Jan. 21, 2016 which is incorporated by reference in its entirety.

### FIELD OF THE INVENTION

The present disclosure is directed to deutero-phenformin and pharmaceutically acceptable salts or solvates thereof, the chemical synthesis thereof, and the medical use of such compounds for the treatment of cancer, diabetes, or polycystic ovarian syndrome.

### BACKGROUND OF INVENTION

Phenformin is a biguanide compound that has been used to treat diabetes, in particular, type 2 diabetes.

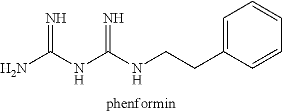 [SMILES: N=C(N)NC(=N)NCCc1ccccc1]

Despite its efficacy as a hypoglycemic agent, phenformin is no longer available for the treatment of diabetes in the United States due to its association with an increased risk of lactic acidosis. Metformin, another biguanide, has replaced phenformin in the United States for the treatment of diabetes. The incidence of lactic acidosis associated with metformin is significantly less than that observed with phenformin.

### SUMMARY OF THE INVENTION

In some aspect, provided herein are compounds of Formula (II) or a pharmaceutically acceptable salt, solvate, or stereoisomer thereof,

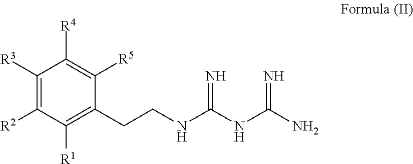

wherein
each of R¹, R², R³, R⁴, and R⁵ is hydrogen or deuterium; wherein at least one R¹, R², R³, R⁴, or R⁵ is deuterium.

In some embodiments of a compound of Formula (II), or a pharmaceutically acceptable salt, solvate, or stereoisomer thereof, R³ is deuterium; and R¹, R², R⁴, and R⁵ are each hydrogen.

In some embodiments of a compound of Formula (II), or a pharmaceutically acceptable salt, solvate, or stereoisomer thereof, R¹, R², R³, R⁴, and R⁵ are all deuterium.

In some embodiments of a compound of Formula (II), or a pharmaceutically acceptable salt, solvate, or stereoisomer thereof, at least two of R¹, R², R³, R⁴, and R⁵ are deuterium.

In some embodiments of a compound of Formula (II), or a pharmaceutically acceptable salt, solvate, or stereoisomer thereof, at least three of R¹, R², R³, R⁴, and R⁵ are deuterium.

In some embodiments of a compound of Formula (II), or a pharmaceutically acceptable salt, solvate, or stereoisomer thereof, R², R³, and R⁴ are deuterium; and R¹ and R⁵ are each hydrogen.

In some embodiments of a compound of Formula (II), or a pharmaceutically acceptable salt, solvate, or stereoisomer thereof, at least four of R¹, R², R³, R⁴, and R⁵ are deuterium.

In another aspect, provided herein is a compound having the structural formula:

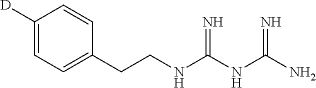 [SMILES: [2H]c1ccc(CCNC(=N)NC(=N)N)cc1]

or a pharmaceutically acceptable salt, solvate, or stereoisomer thereof.

In some embodiments of a compound of Formula (II), or a pharmaceutically acceptable salt, solvate, or stereoisomer thereof, the position represented as D has deuterium enrichment of at least 98%.

In some embodiments of a compound of Formula (II), or a pharmaceutically acceptable salt, solvate, or stereoisomer thereof, the position represented as D has deuterium enrichment of at least 90%.

In some embodiments of a compound of Formula (II), or a pharmaceutically acceptable salt, solvate, or stereoisomer thereof, the position represented as D has deuterium enrichment of at least 50%.

In some embodiments of a compound of Formula (II), or a pharmaceutically acceptable salt, solvate, or stereoisomer thereof, the position represented as D has deuterium enrichment of at least 10%.

In some embodiments of a compound of Formula (II), or a pharmaceutically acceptable salt, solvate, or stereoisomer thereof, the compound has a structural formula selected from the group consisting of:

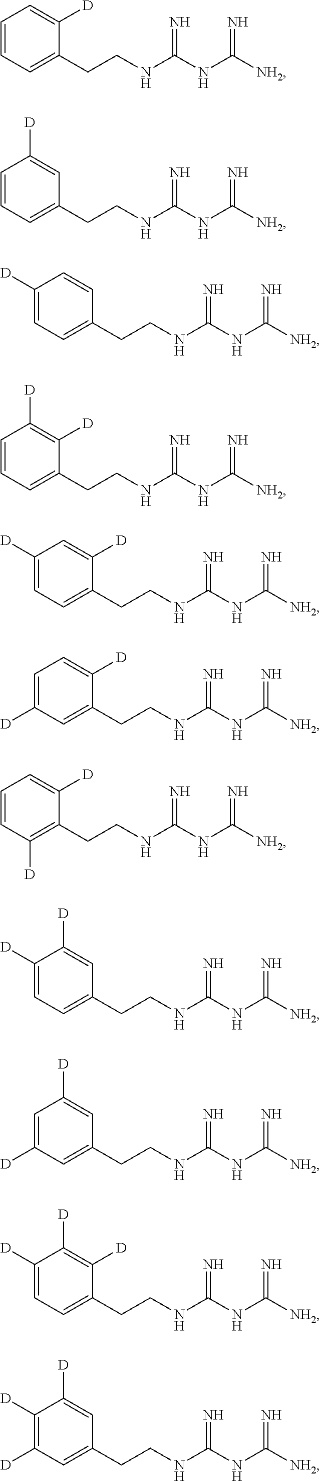

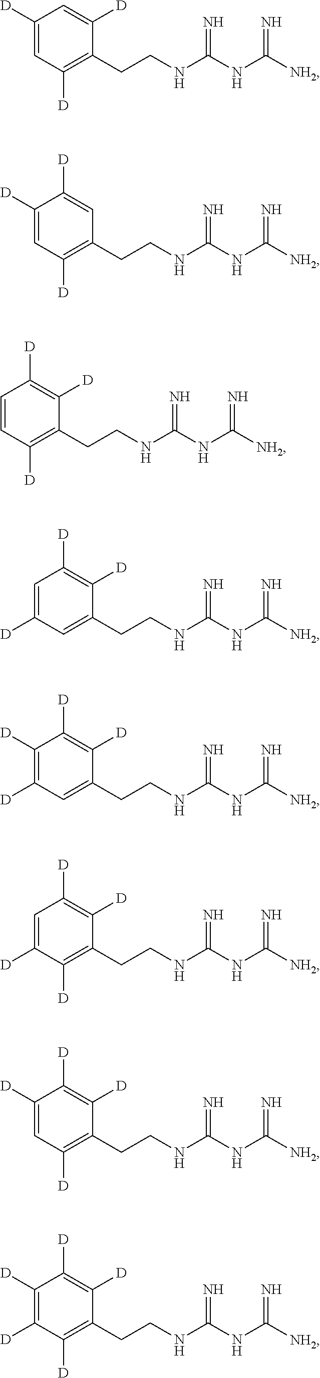

and a pharmaceutically acceptable salt, solvate, or stereoisomer thereof.

In another aspect, provided herein is a pharmaceutical composition comprising a compound of Formula (II), or a pharmaceutically acceptable salt, solvate, or stereoisomer thereof; and a pharmaceutically acceptable carrier thereof.

In another aspect, provided herein is a method of treating cancer in a subject, comprising administering to a subject a therapeutically effective amount of a compound of Formula (I) or a pharmaceutically acceptable salt, solvate, or stereoisomer thereof:

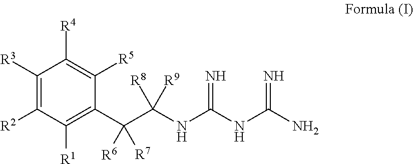

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In some embodiments of a method of treating cancer, the compound of Formula (I) has the structure:

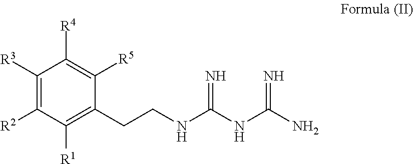

wherein
each of R¹, R², R³, R⁴, and R⁵ is hydrogen or deuterium; wherein at least one R¹, R², R³, R⁴, or R⁵ is deuterium.

In some embodiments of a method of treating cancer, the compound of Formula (II) has the structural formula:

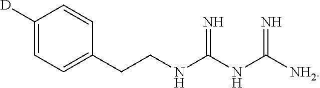 [SMILES: [2H]c1ccc(CCNC(=N)NC(=N)N)cc1]

In some embodiments of a method of treating cancer, the cancer is selected from IDH1 mutant cancers and cancers with LKB1 deficient tumors.

In another aspect, provided herein is a method of treating diabetes in a subject, comprising administering to a subject a therapeutically effective amount of a compound of Formula (I) or a pharmaceutically acceptable salt, solvate, or stereoisomer thereof:

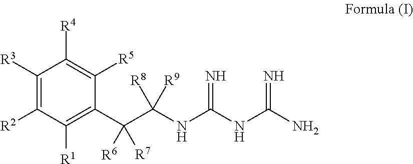

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In some embodiments of a method of treating diabetes, the compound of Formula (I) has the structure:

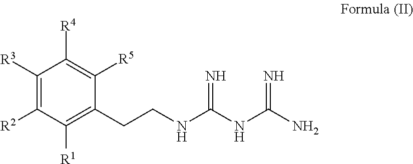

wherein
each of R¹, R², R³, R⁴, and R⁵ is hydrogen or deuterium; wherein at least one R¹, R², R³, R⁴, or R⁵ is deuterium.

In some embodiments of a method of treating diabetes, the compound of Formula (II) has the structural formula:

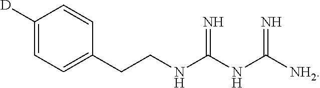 [SMILES: [2H]c1ccc(CCNC(=N)NC(=N)N)cc1]

In some embodiments of a method of treating diabetes, the diabetes is type 2 diabetes.

In another aspect, provided herein is a method of treating polycystic ovarian syndrome in a subject, comprising administering to a subject a therapeutically effective amount of a compound of Formula (I) or a pharmaceutically acceptable salt, solvate, or stereoisomer thereof:

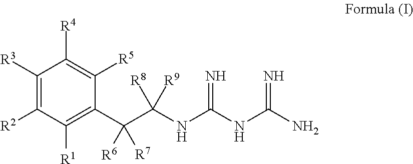

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In some embodiments of a method of treating polycystic ovarian syndrome, the compound of Formula (I) has the structure:

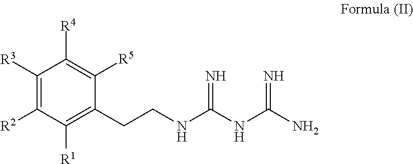

wherein
each of R¹, R², R³, R⁴, and R⁵ is hydrogen or deuterium; wherein at least one R¹, R², R³, R⁴, or R⁵ is deuterium.

In some embodiments of a method of treating polycystic ovarian syndrome, the compound of Formula (II) has the structural formula:

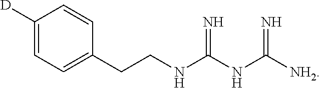 [SMILES: [2H]c1ccc(CCNC(=N)NC(=N)N)cc1]

In some embodiments of a method of treating cancer, diabetes, or polycystic ovarian syndrome the compound has at least one of the following properties:
a) decreased inter-individual variation in plasma levels of the compound or a metabolite there of as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In some embodiments of a method of treating cancer, diabetes, or polycystic ovarian syndrome, the compound has at least two of the following properties:
a) decreased inter-individual variation in plasma levels of the compound or a metabolite there of as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In some embodiments of a method of treating cancer, diabetes, or polycystic ovarian syndrome, the method affects a decreased metabolism of the compound per dosage unit by at least one polymorphically-expressed cytochrome P450 isoform in the subject, as compared to the corresponding non-isotopically enriched compound.

In some embodiments of a method of treating cancer, diabetes, or polycystic ovarian syndrome, the cytochrome P450 isoform is CYP2D6.

In another aspect, provided herein is a method of increasing the bioavailability of a compound of Formula (I) in a subject comprising administering to a subject a compound of Formula (I), or a pharmaceutically acceptable salt, solvate, or stereoisomer thereof:

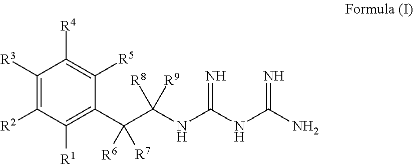

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium;
and a cytochrome P450 inhibitor.

In another aspect, provided herein is a method of decreasing the metabolism of a compound of Formula (I) in a subject comprising administering to a subject a compound of Formula (I), or a pharmaceutically acceptable salt, solvate, or stereoisomer thereof:

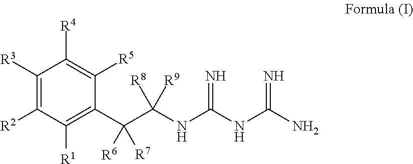

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium;
and a cytochrome P450 inhibitor.

In some embodiments of a method of increasing the bioavailability of a compound of Formula (I) or decreasing the metabolism of a compound of Formula (I), the compound of Formula (I) has the structure:

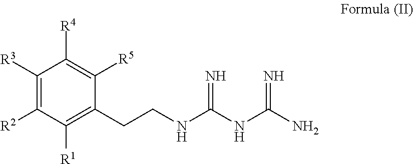

wherein
each of R¹, R², R³, R⁴, and R⁵ is hydrogen or deuterium; wherein at least one R¹, R², R³, R⁴, or R⁵ is deuterium.

In some embodiments of a method of increasing the bioavailability of a compound of Formula (I) or decreasing the metabolism of a compound of Formula (I), the compound of Formula (II) has the structural formula:

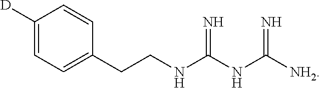 [SMILES: [2H]c1ccc(CCNC(=N)NC(=N)N)cc1]

In some embodiments of a method of increasing the bioavailability of a compound of Formula (I) or decreasing the metabolism of a compound of Formula (I), the cytochrome P450 inhibitor is a CYP2D6 inhibitor.

In some embodiments of a method of increasing the bioavailability of a compound of Formula (I) or decreasing the metabolism of a compound of Formula (I), the CYP2D6 inhibitor is quinidine.

In another aspect, provided herein is a method of increasing the bioavailability of phenformin in a subject comprising administering to a subject phenformin, or a pharmaceutically acceptable salt or solvate thereof and a cytochrome P450 inhibitor.

In another aspect, provided herein is a method of decreasing the metabolism of phenformin in a subject comprising administering to a subject phenformin, or a pharmaceutically acceptable salt or solvate thereof and a cytochrome P450 inhibitor.

In some embodiments of a method of increasing the bioavailability of phenformin or decreasing the metabolism of phenformin, the cytochrome P450 inhibitor is a CYP2D6 inhibitor.

In some embodiments of a method of increasing the bioavailability of phenformin or decreasing the metabolism of phenformin, the CYP2D6 inhibitor is quinidine.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF DRAWINGS

FIG. 1 shows the correlation between CYP 2D6 human polymorph distribution and phenformin efficacy and lactic acidosis risk.

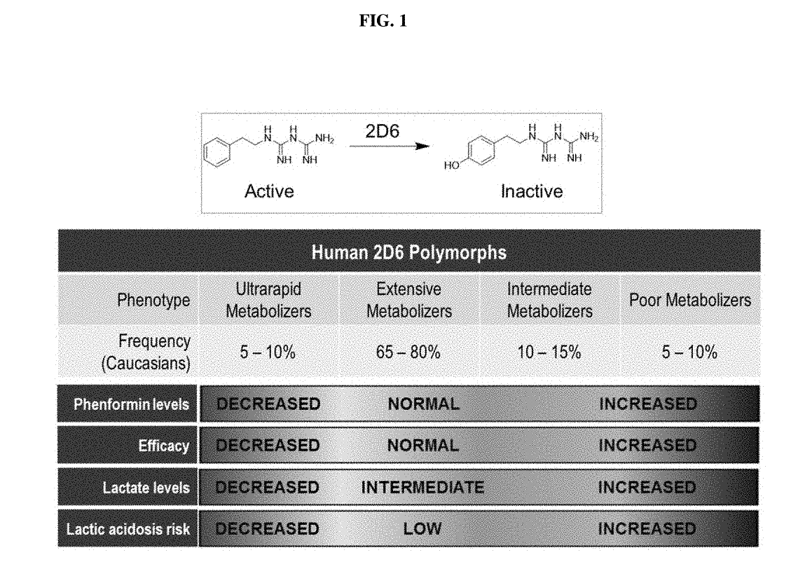

### DETAILED DESCRIPTION

Metformin is an approved type 2 diabetes drug that has demonstrated anti-cancer activity in a number of retrospective analyses. Beginning in 2005 a number of studies have demonstrated improved survival in a range of different cancers including breast, colon, pancreatic, and prostate. However, a number of studies have not found improvements. From this it is clear that although metformin has anti-cancer activities it is unlikely that it is potent enough to offer true benefit to a wide range of cancer patients. Thus, a compound that has similar activities as metformin but more potent would be a significant improvement and offer benefit to many cancer patients. In non-clinical studies, phenformin, a member of the biguanide structural class that includes metformin, consistently demonstrates improved potency and activity against a wider set of cancer types than metformin.

Phenformin was approved to treat type 2 diabetes in 1959, however the FDA rescinded its approval in the United States in 1978 due to its association with an increased risk of lactic acidosis (0.6 events per 1,000 patient years) compared to metformin (0.1 events per 1,000 patient years).

Thus there remains a need to identify compounds that retain phenformin's superior anticancer activity while reducing the risk of lactic acidosis.

Since approval was rescinded in 1978 researchers have demonstrated that phenformin is metabolized by the cytochrome P450 2D6 to an inactive metabolite. A large fraction of the human population has a functional deficiency in the CYP2D6 enzyme that leads to a significant increase in the levels of phenformin in this poor metabolizer population. 2D6 polymorphisms in humans leads to large phenformin metabolism variability ranging from essentially no metabolism in poor metabolizers to 200-fold higher metabolism in ultrarapid metabolizers. It is now recognized that in 2D6 poor metabolizers there is a significant increase in active phenformin levels leading to increased lactate levels that contributes to the development of lactic acidosis and places this patient group at increased risk. In addition there are a significant number of concomitant drugs prescribed to cancer patients that either inhibit or are substrates of 2D6 that could lead to reduced phenformin metabolism further increasing the risk of lactic acidosis in this patient population.

Conversely, the high CYP2D6 metabolizers have low levels of phenformin upon dosing, thereby decreasing the desired efficacy.

Given this it is desirable to provide a compound that has the beneficial activities of phenformin and may also have other benefits including decreased metabolic liability to extend its pharmacological effective life, reduce adverse side effects, to decrease population pharmacokinetic variability, decrease its potential for dangerous drug-drug interactions, or to decrease the risk of phenformin induced lactic acidosis.

To facilitate understanding of the disclosure set forth herein, a number of terms are defined below.

As used herein, the singular forms “a,” “an,” and “the” may refer to plural articles unless specifically stated otherwise. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term herein, those in this section prevail unless stated otherwise.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The terms “prevent,” “preventing,” and “prevention” refer to a method of delaying or precluding the onset of a disorder, disease, or condition; and/or its attendant symptoms, barring a subject from acquiring a disease or reducing a subject's risk of acquiring a disorder, disease, or condition.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulationf, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “deuterium enrichment” refers to the percentage of incorporation of deuterium at a given position in a molecule in the place of hydrogen. For example, deuterium enrichment of about 1% at a given position means that about 1% of molecules in a given sample contain deuterium at the specified position. Because the naturally occurring distribution of deuterium is about 0.0156%, deuterium enrichment at any positions in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% of the molecules are a single compound, including a racemic mixture or single stereoisomer thereof, as determined by standard analytical methods.

The term “about” or “approximately” means an acceptable error for a particular value as determined by one of ordinary skill in the art, which depends in part on how the value is measured or determined. In certain embodiments, “about” can mean with 1 or more standard deviations.

The terms “active ingredient” and “active substance” refer to a compound, which is administered, alone or in combination with one or more pharmaceutically acceptable excipients, to a subject for treating, preventing, or ameliorating one or more symptoms of a disorder or disease.

The terms “drug,” “therapeutic agent,” and “chemotherapeutic agent” refer to a compound, or a pharmaceutical composition thereof, which is administered to a subject for treating, preventing, or ameliorating one or more symptoms of a disorder or disease.

The term “release controlling excipient” refers to an excipient whose primary function is to modify the duration or place of release of the active substance from a dosage form as compared with a conventional immediate release dosage form.

The term “non-release controlling excipient” refers to an excipient whose primary function do not include modifying the duration or place of release of the active substance from a dosage form as compared with a conventional immediate release dosage form.

Deuterium Kinetic Isotope Effect

In an attempt to eliminate foreign substances, such as therapeutic agents, from its circulation system, the animal body expresses various enzymes, such as the cytochrome P₄₅₀ enzymes or CYPs, esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ac^{-Eact/RT}, where E_{act} is the activation energy, T is temperature, R is the molar gas constant, k is the rate constant for the reaction, and A (the frequency factor) is a constant specific to each reaction that depends on the probability that the molecules will collide with the correct orientation. The Arrhenius equation states that the fraction of molecules that have enough energy to overcome an energy barrier, that is, those with energy at least equal to the activation energy, depends exponentially on the ratio of the activation energy to thermal energy (RT), the average amount of thermal energy that molecules possess at a certain temperature.

The transition state in a reaction is a short lived state (on the order of 10⁻¹⁴ sec) along the reaction pathway during which the original bonds have stretched to their limit. By definition, the activation energy E_{act} for a reaction is the energy required to reach the transition state of that reaction. Reactions that involve multiple steps will necessarily have a number of transition states, and in these instances, the activation energy for the reaction is equal to the energy difference between the reactants and the most unstable transition state. Once the transition state is reached, the molecules can either revert, thus reforming the original reactants, or new bonds form giving rise to the products. This dichotomy is possible because both pathways, forward and reverse, result in the release of energy. A catalyst facilitates a reaction process by lowering the activation energy leading to a transition state. Enzymes are examples of biological catalysts that reduce the energy necessary to achieve a particular transition state.

A carbon-hydrogen bond is by nature a covalent chemical bond. Such a bond forms when two atoms of similar electronegativity share some of their valence electrons, thereby creating a force that holds the atoms together. This force or bond strength can be quantified and is expressed in units of energy, and as such, covalent bonds between various atoms can be classified according to how much energy must be applied to the bond in order to break the bond or separate the two atoms.

The bond strength is directly proportional to the absolute value of the ground-state vibrational energy of the bond. This vibrational energy, which is also known as the zero-point vibrational energy, depends on the mass of the atoms that form the bond. The absolute value of the zero-point vibrational energy increases as the mass of one or both of the atoms making the bond increases. Since deuterium (D) has twice the mass of hydrogen (H), it follows that a C—D bond is stronger than the corresponding C—H bond. Compounds with C—D bonds are frequently indefinitely stable in H₂O, and have been widely used for isotopic studies. If a C—H bond is broken during a rate-determining step in a chemical reaction (i.e. the step with the highest transition state energy), then substituting a deuterium for that hydrogen will cause a decrease in the reaction rate and the process will slow down. This phenomenon is known as the Deuterium Kinetic Isotope Effect (DKIE) and can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small size of a hydrogen atom, and occurs because transition states involving a proton can sometimes form in the absence of the required activation energy. A deuterium is larger and statistically has a much lower probability of undergoing this phenomenon. Substitution of tritium for hydrogen results in yet a stronger bond than deuterium and gives numerically larger isotope effects.

Discovered in 1932 by Urey, deuterium (D) is a stable and non-radioactive isotope of hydrogen. It was the first isotope to be separated from its element in pure form and has twice the mass of hydrogen, and makes up about 0.02% of the total mass of hydrogen (in this usage meaning all hydrogen isotopes) on earth. When two deuterium atoms bond with one oxygen, deuterium oxide (D₂O or “heavy water”) is formed. D₂O looks and tastes like H₂O, but has different physical properties. It boils at 101.41° C. and freezes at 3.79° C. Its heat capacity, heat of fusion, heat of vaporization, and entropy are all higher than H₂O. It is more viscous and has different solubilizing properties than H₂O.

When pure D₂O is given to rodents, it is readily absorbed and reaches an equilibrium level that is usually about eighty percent of the concentration that is consumed by the animals. The quantity of deuterium required to induce toxicity is extremely high. When 0% to as much as 15% of the body water has been replaced by D₂O, animals are healthy but are unable to gain weight as fast as the control (untreated) group. When about 15% to about 20% of the body water has been replaced with D₂O, the animals become excitable. When about 20% to about 25% of the body water has been replaced with D₂O, the animals are so excitable that they go into frequent convulsions when stimulated. Skin lesions, ulcers on the paws and muzzles, and necrosis of the tails appear. The animals also become very aggressive; males becoming almost unmanageable. When about 30%, of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Tritium (T) is a radioactive isotope of hydrogen, used in research, fusion reactors, neutron generators and radiopharmaceuticals. Mixing tritium with a phosphor provides a continuous light source, a technique that is commonly used in wristwatches, compasses, rifle sights and exit signs. It was discovered by Rutherford, Oliphant and Harteck in 1934, and is produced naturally in the upper atmosphere when cosmic rays react with H₂ molecules. Tritium is a hydrogen atom that has 2 neutrons in the nucleus and has an atomic weight close to 3. It occurs naturally in the environment in very low concentrations, most commonly found as T₂O, a colorless and odorless liquid. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk.

Deuteration of pharmaceuticals to improve pharmacokinetics (PK), pharmacodynamics (PD), and toxicity profiles, has been demonstrated previously with some classes of drugs. For example, DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching which may even give rise to an oxidative intermediate with a faster off-rate from an activating Phase I enzyme (e.g., cytochrome P₄₅₀ 3A4). The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity. Such have not been heretofore sufficiently predictable a priori for any drug class.

Deutero-Phenformin Derivatives

The carbon-hydrogen bonds of phenformin contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation produce a detectable Kinetic Isotope Effect (KIE) that could affect the pharmacokinetic, pharmacologic and/or toxicologic parameters of such hypnotic agents in comparison to compounds having naturally occurring levels of deuterium.

Aspects of the present disclosure describe an approach to design and synthesize new derivatives of phenformin through chemical modifications and derivations of the carbon-hydrogen bonds.

Because the polymorphically expressed CYP 2D6 oxidizes phenformin, the prevention of such interactions decreases interpatient variability, decreases drug-drug interactions, increases T_{1/2}, decreases the necessary Cₘₐₓ, decreases toxicity and risk of lactic acidosis, and improves several other ADMET parameters. Various deuteration patterns can be used to a) reduce or eliminate unwanted metabolites, b) increase the half-life of the parent drug, c) decrease the number of doses needed to achieve a desired effect, d) decrease the amount of a dose needed to achieve a desired effect, e) increase the formation of active metabolites, if any are formed, f) decrease phenformin inter-patient variability, g) decrease the risk of lactic acidosis and/or h) decrease the production of deleterious metabolites in specific tissues and/or create a more effective drug and/or a safer drug for polypharmacy, whether the polypharmacy be intentional or not. The deuteration approach has strong potential to slow the metabolism through the genetically polymorphically expressed CYPs.

In some aspect, provided herein are compounds of Formula (I) or a pharmaceutically acceptable salt, solvate, or stereoisomer thereof:

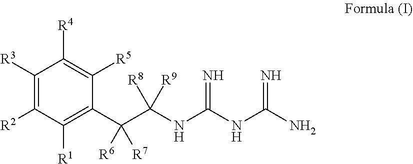

wherein
each of R¹, R², R³, R⁴, R⁵, R⁶, R⁷, R⁸, and R⁹ is hydrogen or deuterium, wherein at least one of R¹, R², R³, R⁴, R⁵, R⁶, R⁷, R⁸, or R⁹ is deuterium.

In some embodiments of a compound of Formula (I) or a pharmaceutically acceptable salt, solvate, or stereoisomer thereof, at least two of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least three of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least four of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least five of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least six of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least seven of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, at least eight of R¹, R², R³, R⁴, R⁵, R⁶, R⁷, R⁸, or R⁹ are deuterium.

In some embodiments of a compound of Formula (I) or a pharmaceutically acceptable salt, solvate, or stereoisomer thereof, R¹, R², R³, R⁴, R⁵, R⁶, R⁷, R⁸, and R⁹ are deuterium.

In some embodiments of a compound of Formula (I) or a pharmaceutically acceptable salt, solvate, or stereoisomer thereof, at least one of R¹, R², R³, R⁴, or R⁵ is deuterium.

In some embodiments of a compound of Formula (I) or a pharmaceutically acceptable salt, solvate, or stereoisomer thereof, at least two of R¹, R², R³, R⁴, or R⁵ are deuterium.

In some embodiments of a compound of Formula (I) or a pharmaceutically acceptable salt, solvate, or stereoisomer thereof, at least three of R¹, R², R³, R⁴, or R⁵ are deuterium.

In some embodiments of a compound of Formula (I) or a pharmaceutically acceptable salt, solvate, or stereoisomer thereof, at least four of R¹, R², R³, R⁴, or R⁵ are deuterium.

In some embodiments of a compound of Formula (I) or a pharmaceutically acceptable salt, solvate, or stereoisomer thereof, R¹, R², R³, R⁴, or R⁵ are deuterium.

In some embodiments of a compound of Formula (I) or a pharmaceutically acceptable salt, solvate, or stereoisomer thereof, at least one of R⁶, R⁷, R⁸, or R⁹ is deuterium.

In some embodiments of a compound of Formula (I) or a pharmaceutically acceptable salt, solvate, or stereoisomer thereof, at least two of R⁶, R⁷, R⁸, or R⁹ are deuterium.

In some embodiments of a compound of Formula (I) or a pharmaceutically acceptable salt, solvate, or stereoisomer thereof, at least three of R⁶, R⁷, R⁸, or R⁹ are deuterium.

In some embodiments of a compound of Formula (I) or a pharmaceutically acceptable salt, solvate, or stereoisomer thereof, R⁶, R⁷, R⁸, and R⁹ are deuterium.

In some embodiments of a compound of Formula (I) or a pharmaceutically acceptable salt, solvate, or stereoisomer thereof, at least one of R¹, R², R³, R⁴, or R⁵ is deuterium and at least one of R⁶, R⁷, R⁸, or R⁹ is deuterium.

In some embodiments of a compound of Formula (I) or a pharmaceutically acceptable salt, solvate, or stereoisomer thereof, at least two of R¹, R², R³, R⁴, or R⁵ are deuterium and at least one of R⁶, R⁷, R⁸, or R⁹ is deuterium.

In some embodiments of a compound of Formula (I) or a pharmaceutically acceptable salt, solvate, or stereoisomer thereof, at least three of R¹, R², R³, R⁴, or R⁵ are deuterium and at least one of R⁶, R⁷, R⁸, or R⁹ is deuterium.

In some embodiments of a compound of Formula (I) or a pharmaceutically acceptable salt, solvate, or stereoisomer thereof, at least four of R¹, R², R³, R⁴, or R⁵ are deuterium and at least one of R⁶, R⁷, R⁸, or R⁹ is deuterium.

In some embodiments of a compound of Formula (I) or a pharmaceutically acceptable salt, solvate, or stereoisomer thereof, R¹, R², R³, R⁴, and R⁵ are deuterium and at least one of R⁶, R⁷, R⁸, or R⁹ is deuterium.

In some embodiments of a compound of Formula (I) or a pharmaceutically acceptable salt, solvate, or stereoisomer thereof, at least one of R¹, R², R³, R⁴, or R⁵ is deuterium and at least two of R⁶, R⁷, R⁸, or R⁹ are deuterium.

In some embodiments of a compound of Formula (I) or a pharmaceutically acceptable salt, solvate, or stereoisomer thereof, at least one of R¹, R², R³, R⁴, or R⁵ is deuterium and at least three of R⁶, R⁷, R⁸, or R⁹ are deuterium.

In some embodiments of a compound of Formula (I) or a pharmaceutically acceptable salt, solvate, or stereoisomer thereof, at least one of R¹, R², R³, R⁴, or R⁵ is deuterium and R⁶, R⁷, R⁸, and R⁹ are deuterium.

In some embodiments of a compound of Formula (I) or a pharmaceutically acceptable salt, solvate, or stereoisomer thereof, is a compound of Formula (II) or a pharmaceutically acceptable salt, solvate, or stereoisomer thereof:

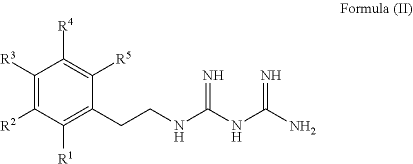

wherein
each of R¹, R², R³, R⁴, and R⁵ is hydrogen or deuterium; wherein at least one R¹, R², R³, R⁴, or R⁵ is deuterium.

In some embodiments of a compound of Formula (II) or a pharmaceutically acceptable salt, solvate, or stereoisomer thereof, R³ is deuterium and R¹, R², R⁴, and R⁵ are each hydrogen.

In some embodiments of a compound of Formula (II) or a pharmaceutically acceptable salt, solvate, or stereoisomer thereof, R¹, R², R³, R⁴, and R⁵ are all deuterium.

In some embodiments of a compound of Formula (II) or a pharmaceutically acceptable salt, solvate, or stereoisomer thereof, at least two of R¹, R², R³, R⁴, and R⁵ are deuterium.

In some embodiments of a compound of Formula (II) or a pharmaceutically acceptable salt, solvate, or stereoisomer thereof, at least three of R¹, R², R³, R⁴, and R⁵ are deuterium.

In some embodiments of a compound of Formula (II) or a pharmaceutically acceptable salt, solvate, or stereoisomer thereof, R², R³, R⁴ are deuterium and R¹ and R⁵ are each hydrogen.

In some embodiments of a compound of Formula (II) or a pharmaceutically acceptable salt, solvate, or stereoisomer thereof, at least four of R¹, R², R³, R⁴, and R⁵ are deuterium.

In another aspect, disclosed herein is a compound of Formula (I) or Formula (II) having a structural formula selected from the group consisting of:

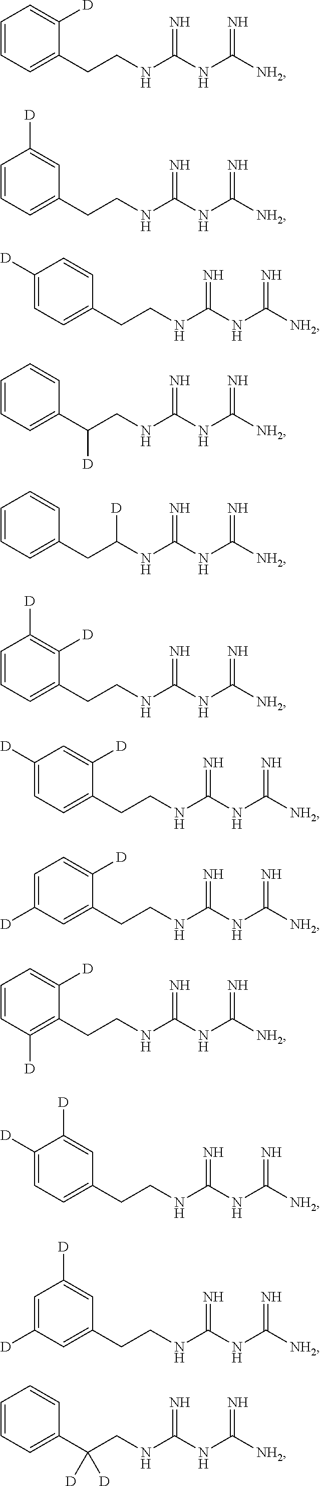

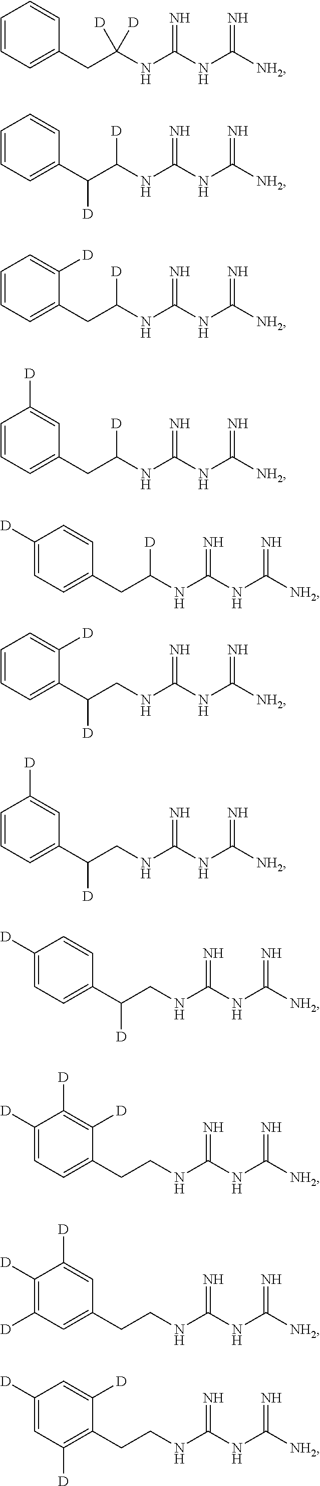

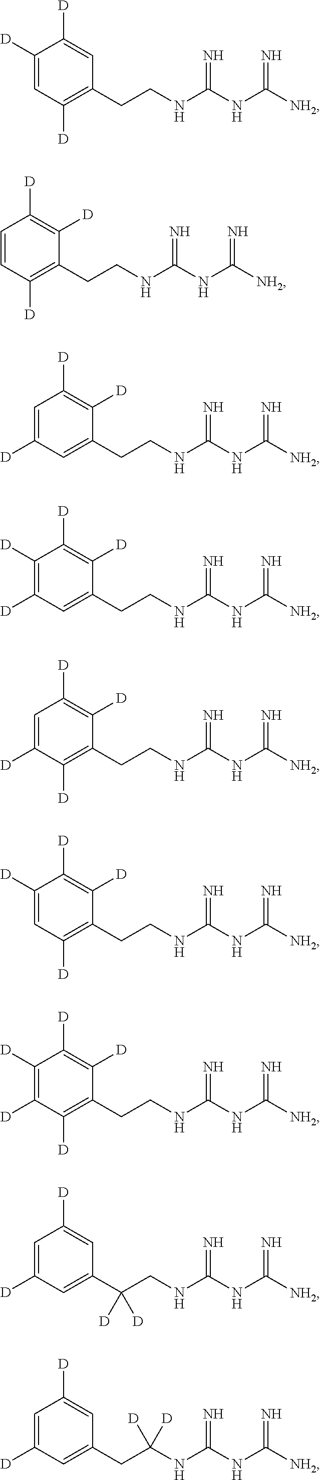

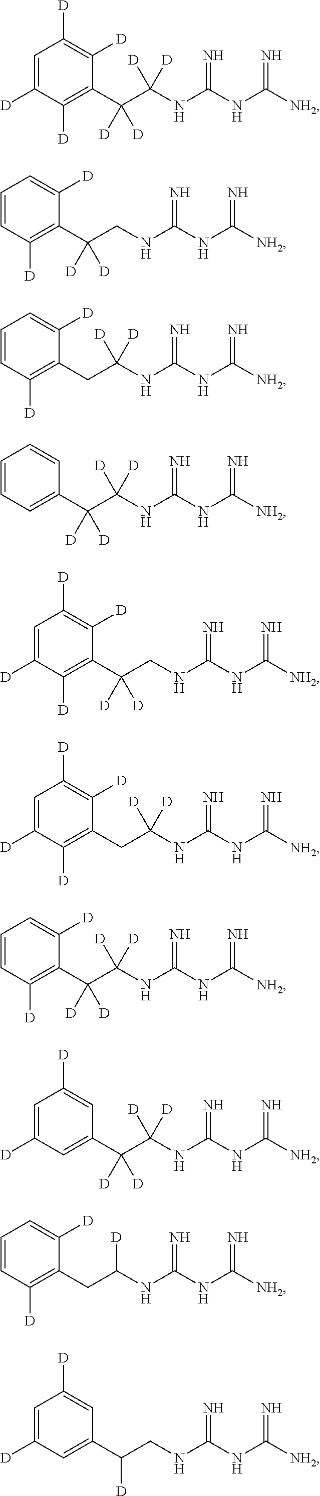

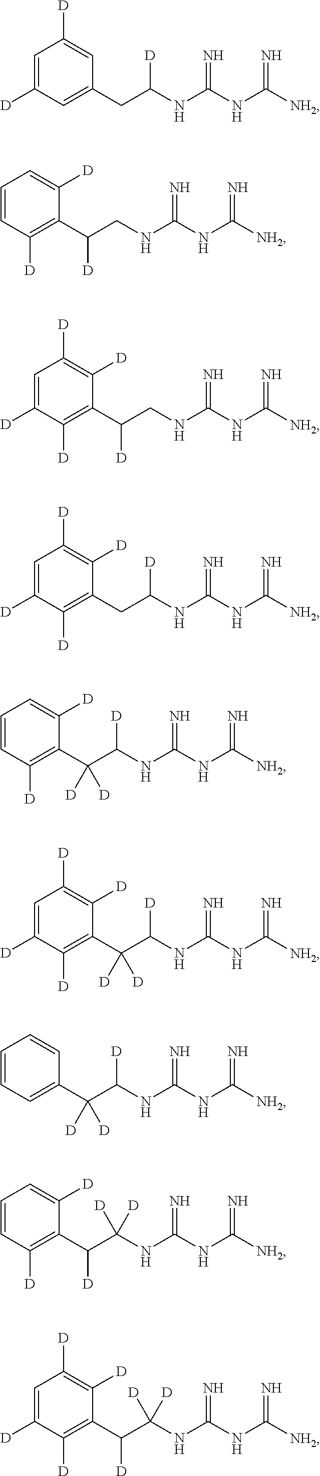

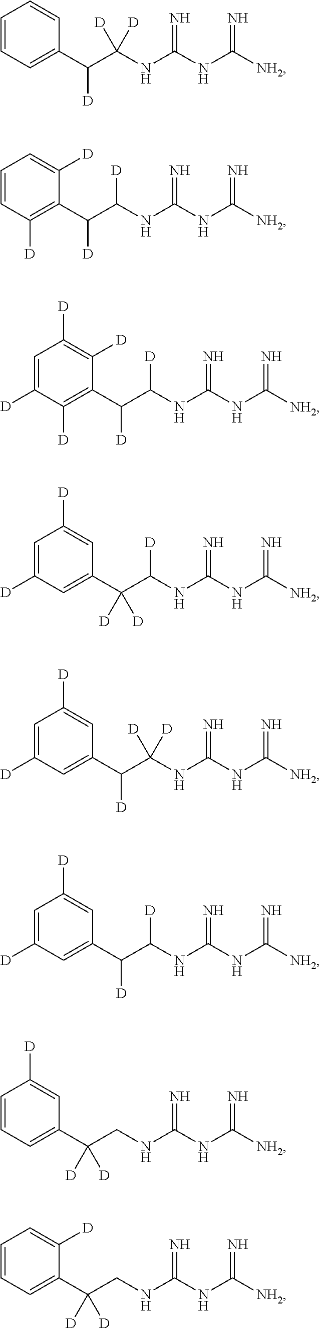

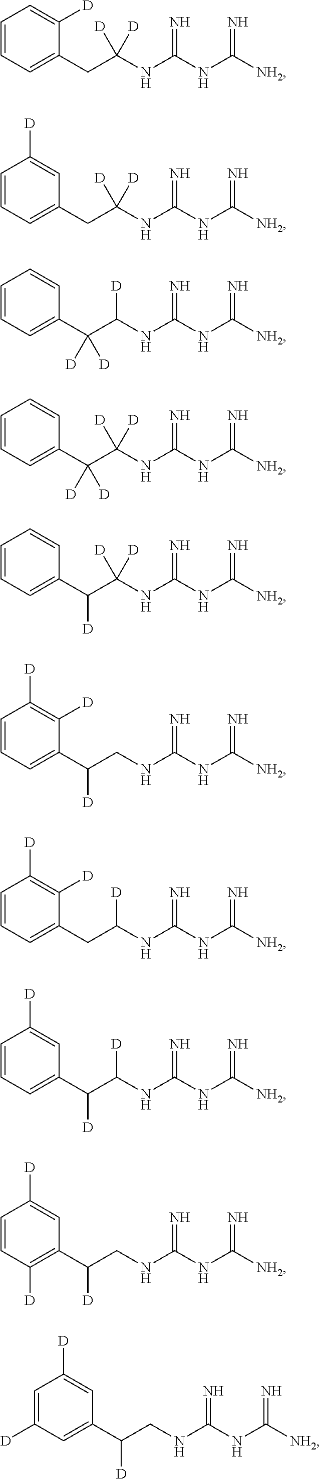

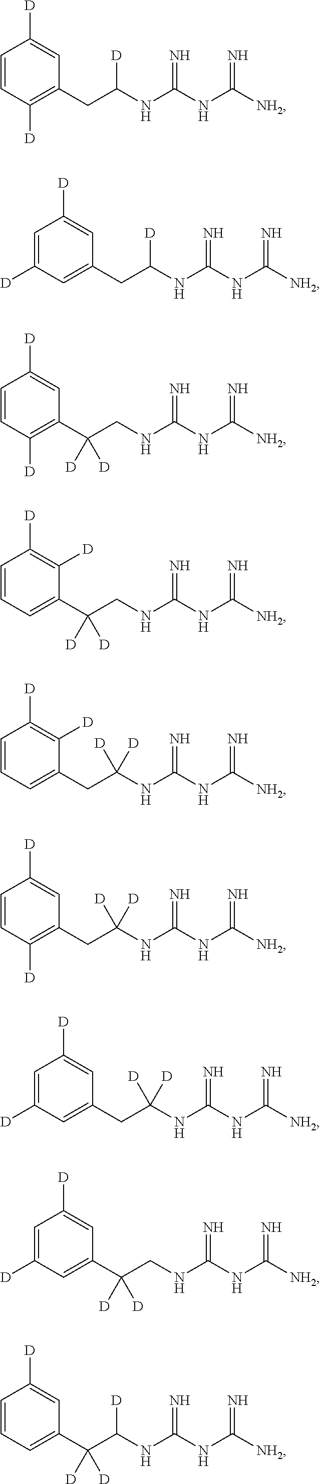

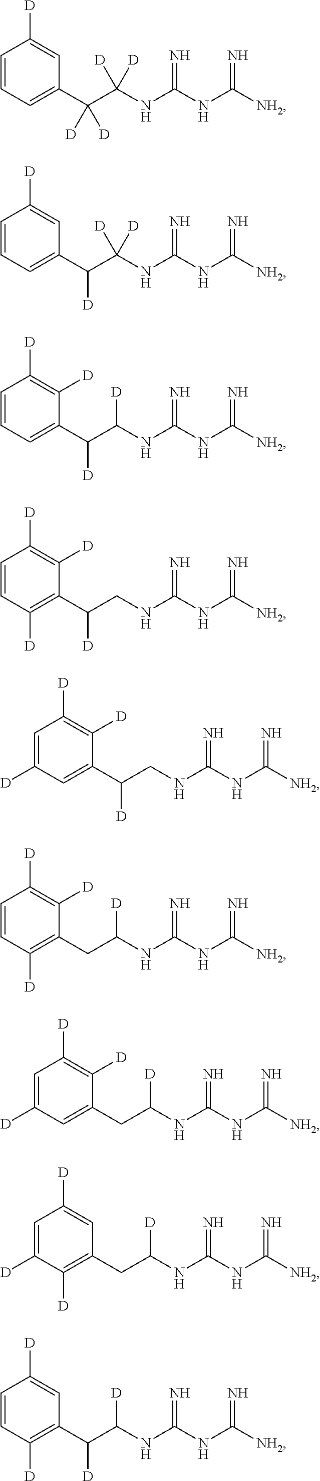

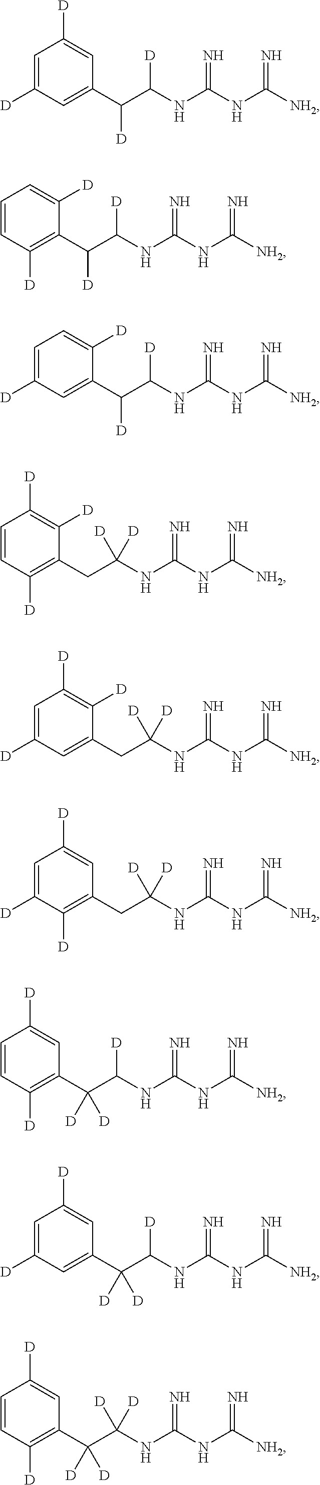

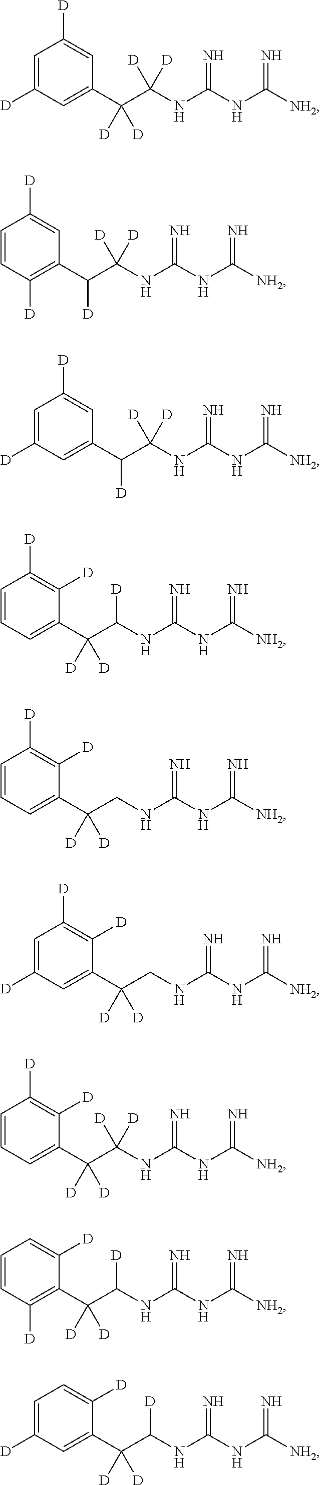

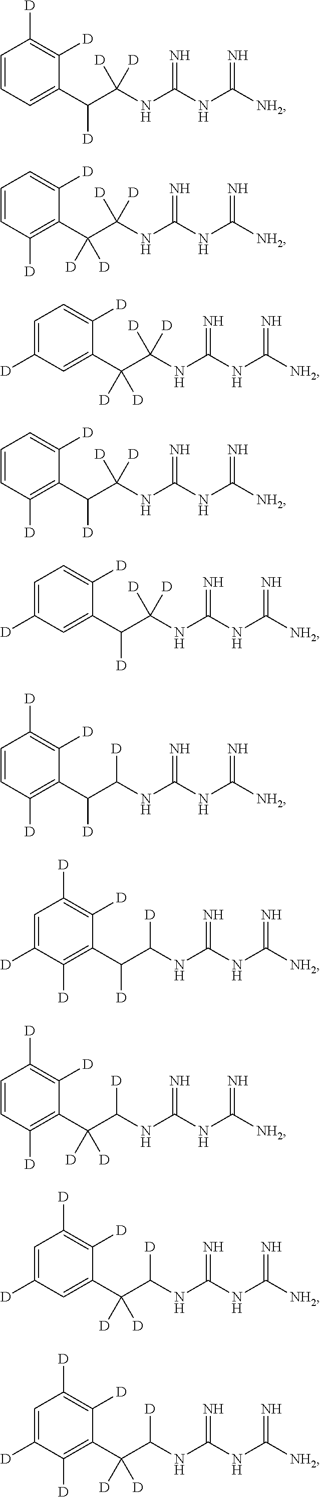

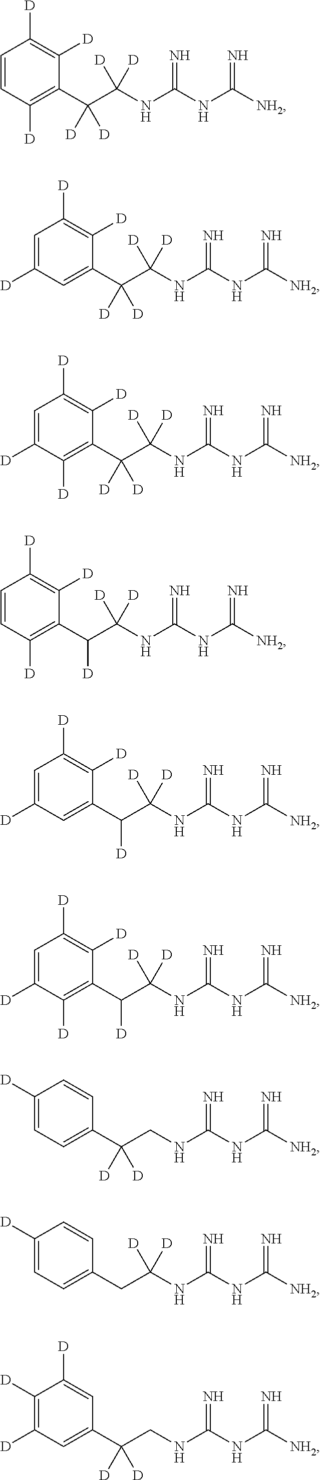

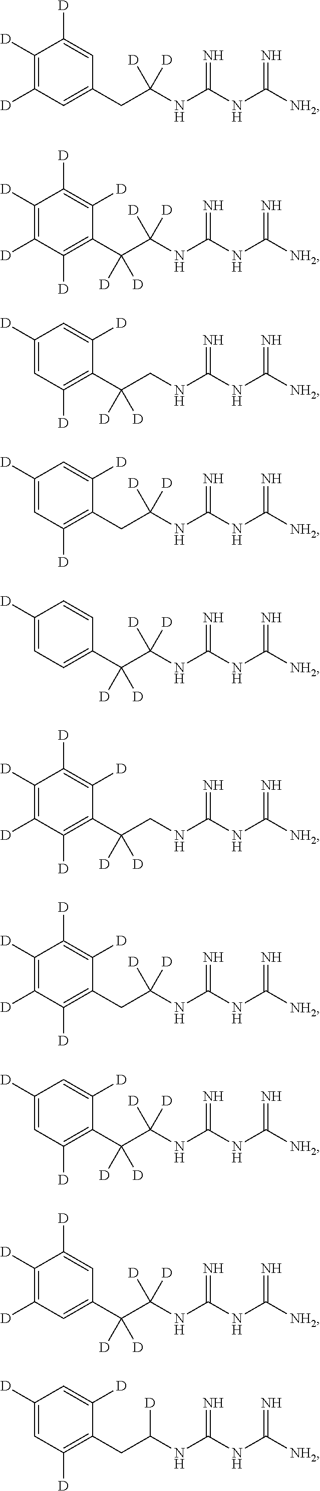

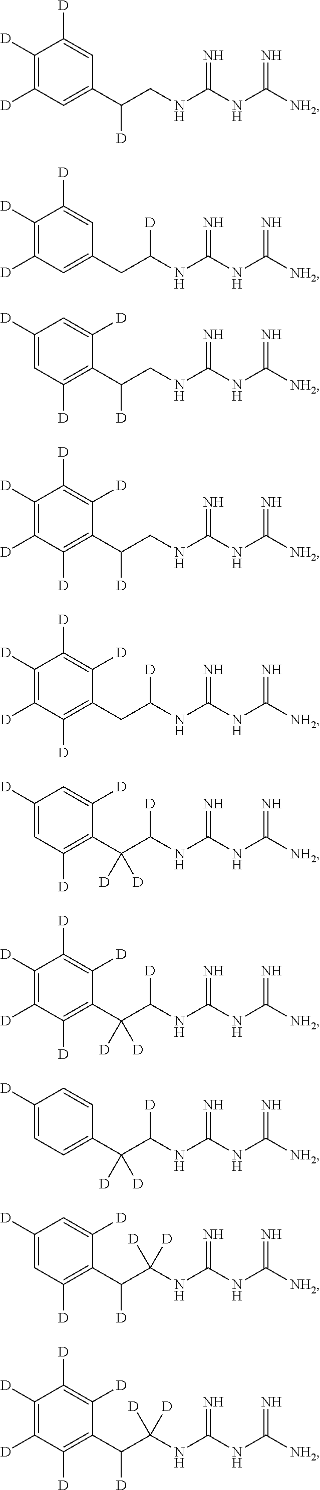

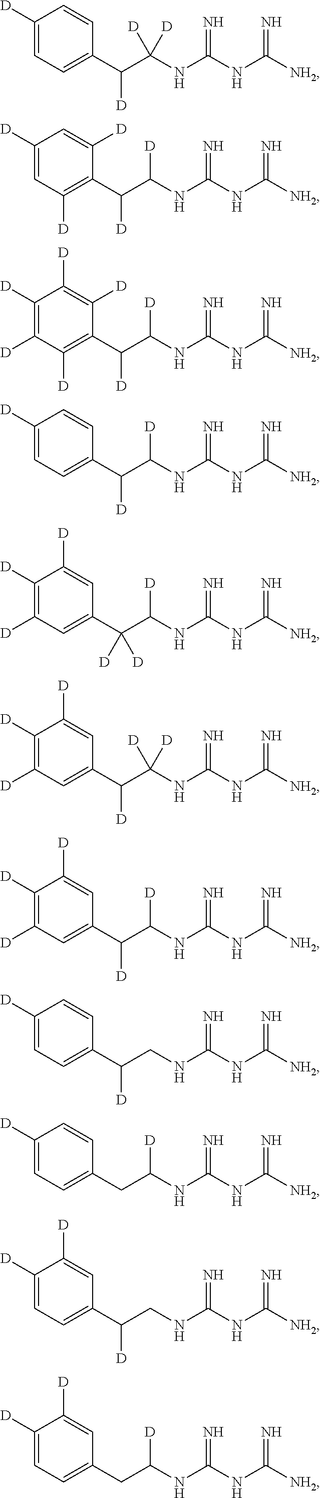

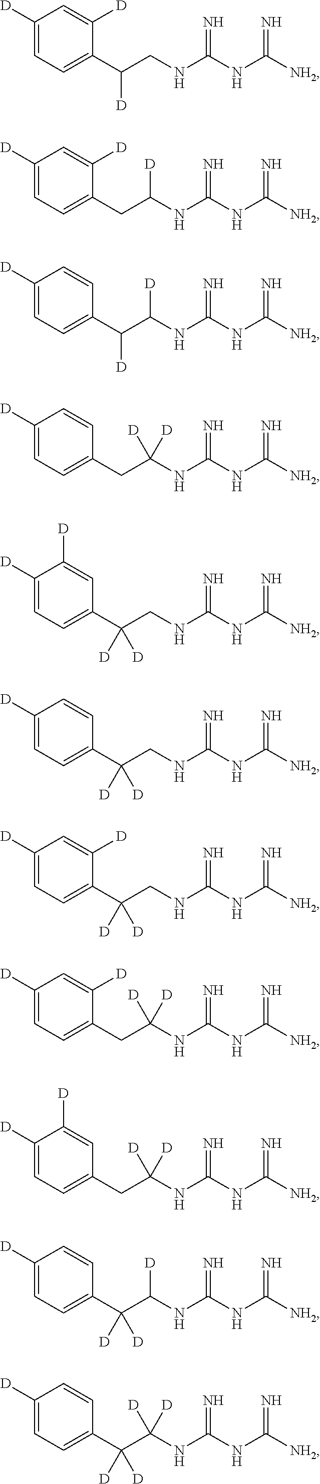

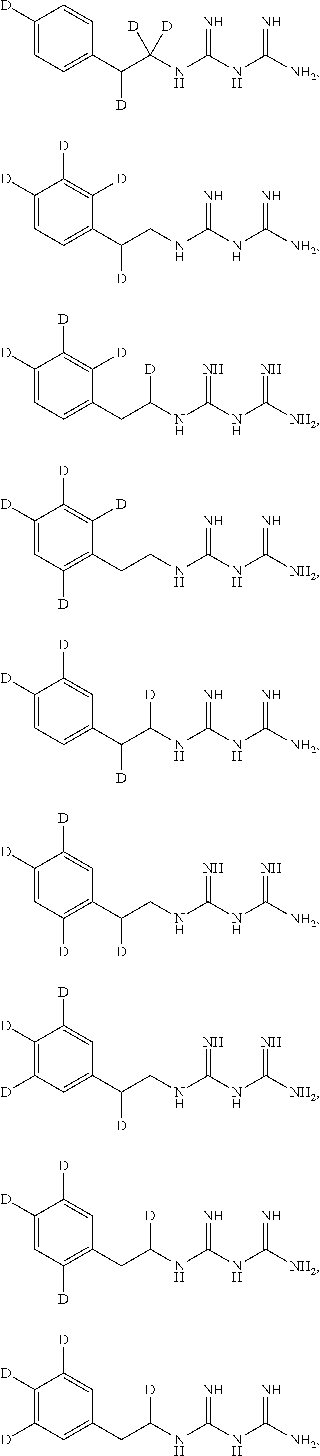

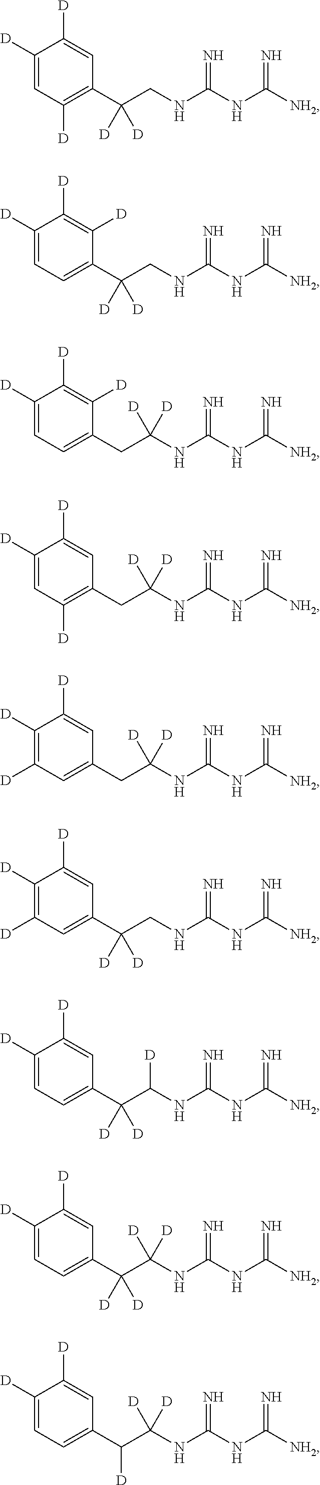

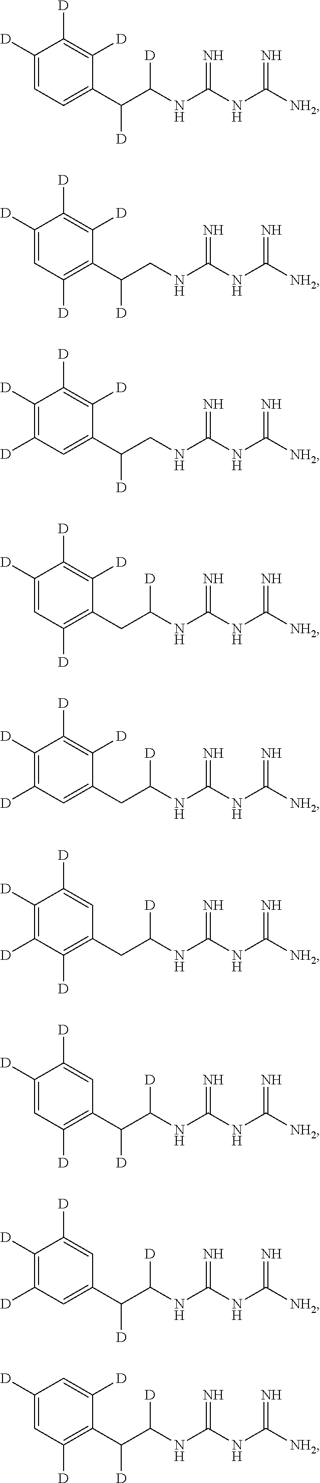

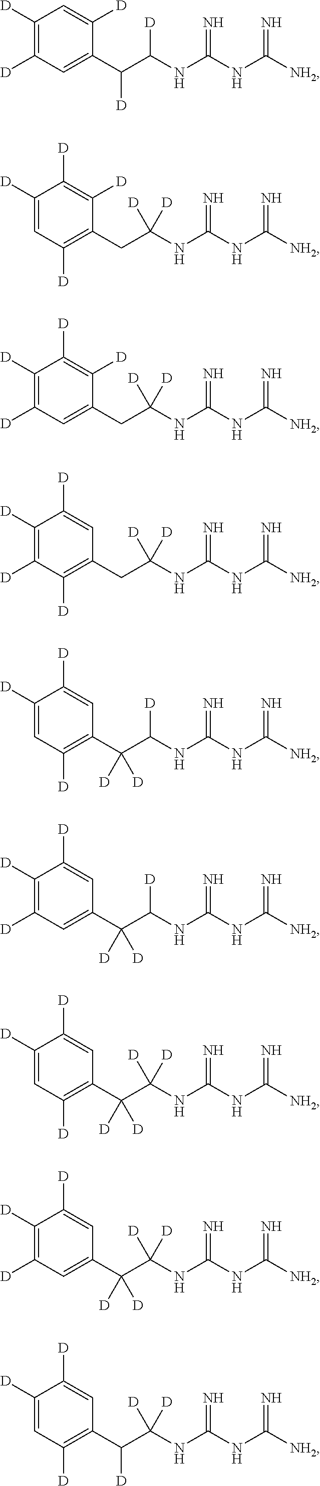

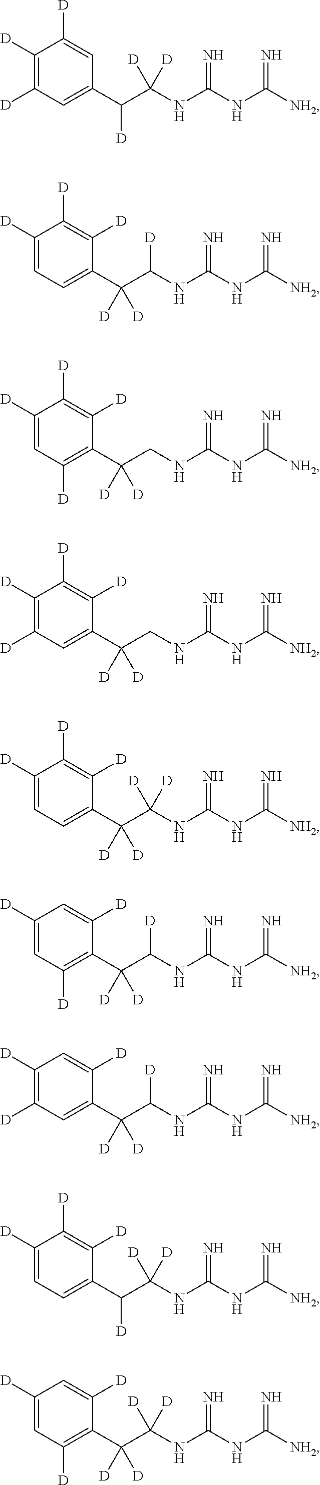

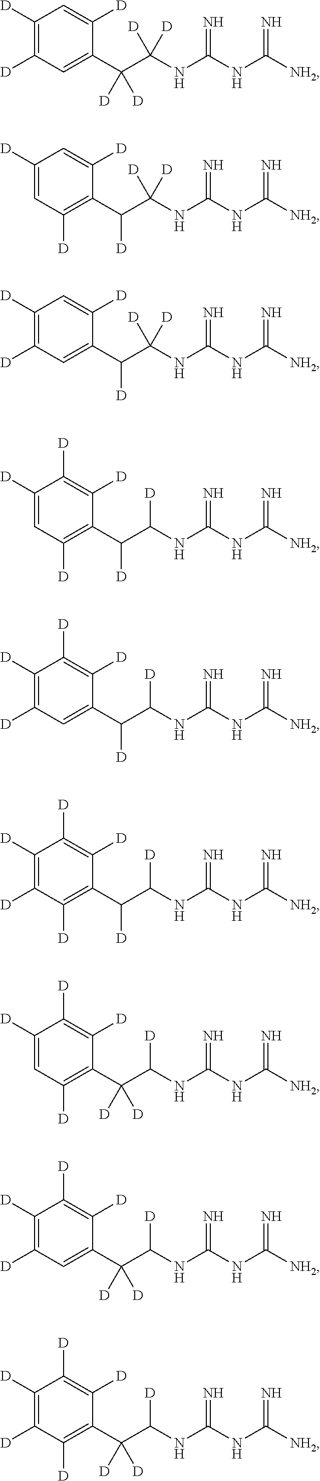

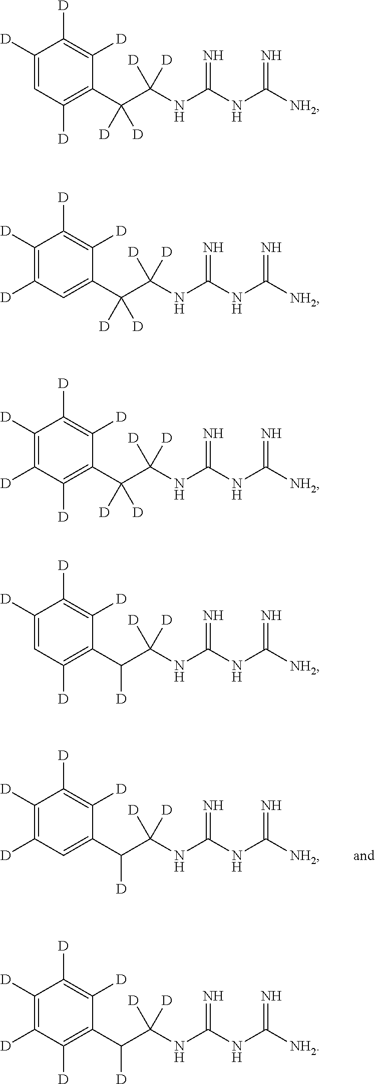 [SMILES: [2H]c1cc([2H])c(C([2H])([2H])C([2H])([2H])NC(=N)NC(=N)N)c([2H])c1[2H] ; [2H]c1cc(C([2H])([2H])C([2H])([2H])NC(=N)NC(=N)N)c([2H])c([2H])c1[2H] ; [2H]c1c([2H])c([2H])c(C([2H])([2H])C([2H])([2H])NC(=N)NC(=N)N)c([2H])c1[2H] ; [2H]c1cc([2H])c(C([2H])C([2H])([2H])NC(=N)NC(=N)N)c([2H])c1[2H] ; [2H]c1cc(C([2H])C([2H])([2H])NC(=N)NC(=N)N)c([2H])c([2H])c1[2H] ; [2H]c1c([2H])c([2H])c(C([2H])C([2H])([2H])NC(=N)NC(=N)N)c([2H])c1[2H]]

In some embodiments of a compound of Formula (II) or a pharmaceutically acceptable salt, solvate, or stereoisomer thereof, the compound has a structural formula selected from the group consisting of:

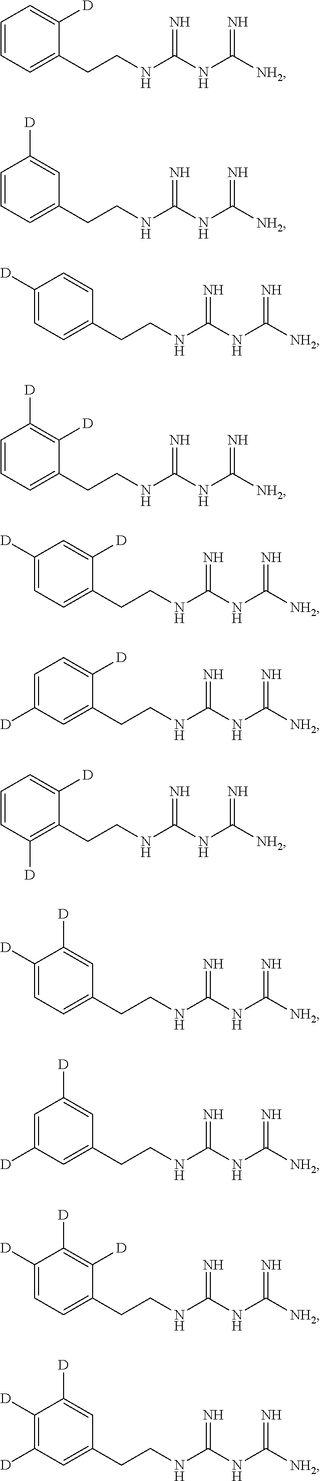

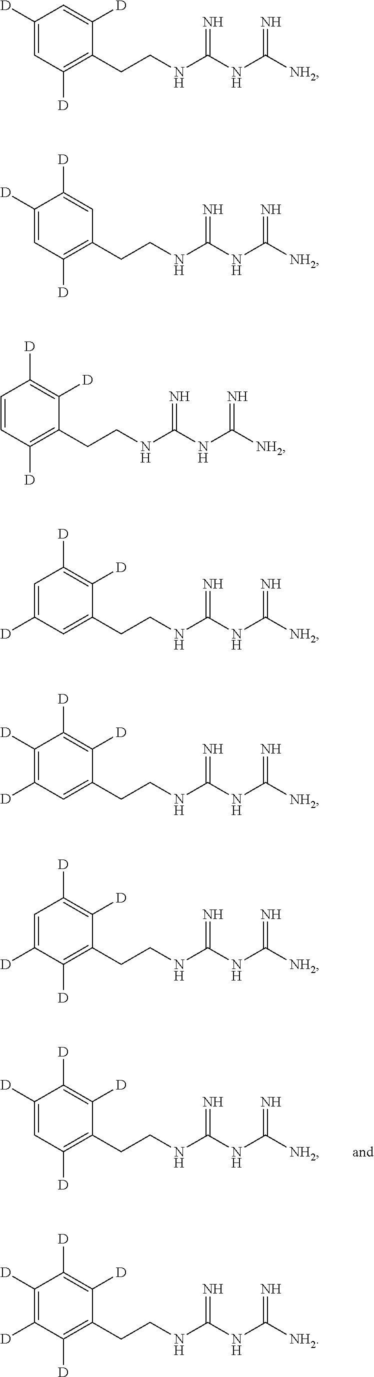

In some embodiments of a compound of Formula (II) or a pharmaceutically acceptable salt, solvate, or stereoisomer thereof, the compound has the structural formula:

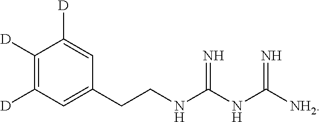 [SMILES: [2H]c1cc(CCNC(=N)NC(=N)N)cc([2H])c1[2H]]

In some embodiments of a compound of Formula (II) or a pharmaceutically acceptable salt, solvate, or stereoisomer thereof, the compound has the structural formula:

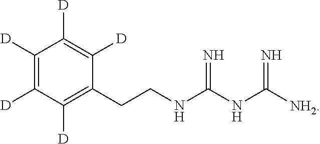 [SMILES: [2H]c1c([2H])c([2H])c(CCNC(=N)NC(=N)N)c([2H])c1[2H]]

In some embodiments of a compound of Formula (II) or a pharmaceutically acceptable salt, solvate, or stereoisomer thereof, the compound has the structural formula:

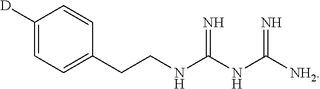 [SMILES: [2H]c1ccc(CCNC(=N)NC(=N)N)cc1]

In some embodiments of a compound of Formula (II) or a pharmaceutically acceptable salt, solvate, or stereoisomer thereof, the compound has the structural formula:

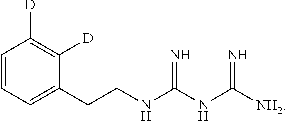 [SMILES: [2H]c1cccc(CCNC(=N)NC(=N)N)c1[2H]]

In some embodiments of a compound of Formula (II) or a pharmaceutically acceptable salt, solvate, or stereoisomer thereof, the compound has the structural formula:

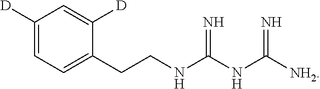 [SMILES: [2H]c1ccc(CCNC(=N)NC(=N)N)c([2H])c1]

In some embodiments of a compound of Formula (II) or a pharmaceutically acceptable salt, solvate, or stereoisomer thereof, the compound has the structural formula:

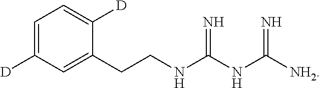 [SMILES: [2H]c1ccc([2H])c(CCNC(=N)NC(=N)N)c1]

In some embodiments of a compound of Formula (II) or a pharmaceutically acceptable salt, solvate, or stereoisomer thereof, the compound has the structural formula:

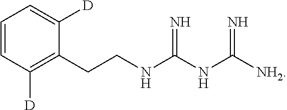 [SMILES: [2H]c1cccc([2H])c1CCNC(=N)NC(=N)N]

In some embodiments of a compound of Formula (II) or a pharmaceutically acceptable salt, solvate, or stereoisomer thereof, the compound has the structural formula:

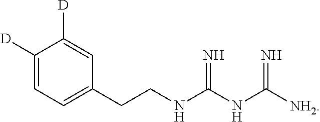 [SMILES: [2H]c1ccc(CCNC(=N)NC(=N)N)cc1[2H]]

In some embodiments of a compound of Formula (II) or a pharmaceutically acceptable salt, solvate, or stereoisomer thereof, the compound has the structural formula:

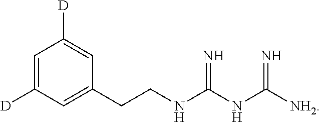 [SMILES: [2H]c1cc([2H])cc(CCNC(=N)NC(=N)N)c1]

In some embodiments of a compound of Formula (I) or Formula (II) or a pharmaceutically acceptable salt, solvate, or stereoisomer thereof, any position represented as D has deuterium enrichment of at least 98%.

In some embodiments of a compound of Formula (I) or Formula (II) or a pharmaceutically acceptable salt, solvate, or stereoisomer thereof, any position represented as D has deuterium enrichment of at least 90%.

In some embodiments of a compound of Formula (I) or Formula (II) or a pharmaceutically acceptable salt, solvate, or stereoisomer thereof, any position represented as D has deuterium enrichment of at least 80%.

In some embodiments of a compound of Formula (I) or Formula (II) or a pharmaceutically acceptable salt, solvate, or stereoisomer thereof, any position represented as D has deuterium enrichment of at least 70%.

In some embodiments of a compound of Formula (I) or Formula (II) or a pharmaceutically acceptable salt, solvate, or stereoisomer thereof, any position represented as D has deuterium enrichment of at least 60%.

In some embodiments of a compound of Formula (I) or Formula (II) or a pharmaceutically acceptable salt, solvate, or stereoisomer thereof, any position represented as D has deuterium enrichment of at least 50%.

In some embodiments of a compound of Formula (I) or Formula (II) or a pharmaceutically acceptable salt, solvate, or stereoisomer thereof, any position represented as D has deuterium enrichment of at least 40%.

In some embodiments of a compound of Formula (I) or Formula (II) or a pharmaceutically acceptable salt, solvate, or stereoisomer thereof, any position represented as D has deuterium enrichment of at least 30%.

In some embodiments of a compound of Formula (I) or Formula (II) or a pharmaceutically acceptable salt, solvate, or stereoisomer thereof, any position represented as D has deuterium enrichment of at least 20%.

In some embodiments of a compound of Formula (I) or Formula (II) or a pharmaceutically acceptable salt, solvate, or stereoisomer thereof, any position represented as D has deuterium enrichment of at least 10%.

In some embodiments of a compound of Formula (I) or Formula (II) or a pharmaceutically acceptable salt, solvate, or stereoisomer thereof, any position represented as D has deuterium enrichment of at least 5%.

In some embodiments of a compound of Formula (I) or Formula (II) or a pharmaceutically acceptable salt, solvate, or stereoisomer thereof, any position represented as D has deuterium enrichment of at least 1%.

In some embodiments, the deuterated compound of Formula (I) or Formula (II) also contain less prevalent isotopes for other elements, including, but not limited to, ¹³C or ¹⁴C for carbon and ¹⁵N for nitrogen.

In one embodiment, the deuterated compounds provided herein maintain the beneficial aspects of the corresponding non-isotopically enriched molecules while substantially decreasing toxicity (reducing the risk of lactic acidosis), increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Isotopic hydrogen can be introduced into a compound of Formula (I) or Formula (II) as provided herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. In addition, the molecule being labeled may be changed, depending upon the severity of the synthetic reaction employed. Exchange techniques, on the other hand, may yield lower tritium or deuterium incorporation, often with the isotope being distributed over many sites on the molecule, but offer the advantage that they do not require separate synthetic steps and are less likely to disrupt the structure of the molecule being labeled.

The compounds of Formula (I) or Formula (II) as provided herein can be prepared by methods known to one of skill in the art or following procedures similar to those described in the Example section herein and routine modifications thereof. For an example, the compound of Formula (I) or Formula (II) can be prepared as shown in Scheme 1.

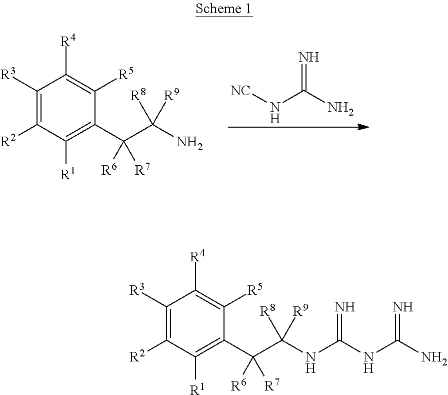

Deuterium can be incorporated to different positions synthetically by using appropriate deuterated intermediates. These deuterated intermediates are either commercially available, or can be prepared by methods known to one of skill in the art or following procedures similar to those described in the Journal of Organic Chemistry, 48(20), 3458-3464, 1983 or Journal of Chemical and Engineering Data, 55(5), 2048-2054; 2010 and routine modifications thereof.

It is to be understood that the compounds provided herein may contain one or more chiral centers, chiral axes, and/or chiral planes, as described in “Stereochemistry of Carbon Compounds” Eliel and Wilen, John Wiley & Sons, New York, 1994, pp. 1119-1190. Such chiral centers, chiral axes, and chiral planes may be of either the (R) or (S) configuration, or may be a mixture thereof.

Another method for characterizing a composition containing a compound having at least one chiral center is by the effect of the composition on a beam of polarized light. When a beam of plane polarized light is passed through a solution of a chiral compound, the plane of polarization of the light that emerges is rotated relative to the original plane. This phenomenon is known as optical activity, and compounds that rotate the plane of polarized light are said to be optically active. One enantiomer of a compound will rotate the beam of polarized light in one direction, and the other enantiomer will rotate the beam of light in the opposite direction. The enantiomer that rotates the polarized light in the clockwise direction is the (+)-enantiomer, and the enantiomer that rotates the polarized light in the counterclockwise direction is the (−)-enantiomer.

The compounds provided herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

In some embodiments, the compound of Formula (I) or Formula (II) are provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

The compound of Formula (I) or Formula (II) may also be provided as a prodrug, which is a functional derivative of the compound of Formula (I) or Formula (II) and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev.1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Pharmaceutical Compositions

Provided herein are pharmaceutical compositions comprising a compound of Formula (I) or Formula (II), or a pharmaceutically acceptable salt, solvate, or stereoisomer thereof, in a pharmaceutically acceptable vehicle, carrier, diluent, or excipient, or a mixture thereof; and one or more pharmaceutically acceptable excipients or carriers.

Also provided herein are pharmaceutical compositions in modified release dosage forms, which comprise a compound of Formula (I) or Formula (II), or a pharmaceutically acceptable salt, solvate, or stereoisomer thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

Further, provided herein are pharmaceutical compositions in enteric coated dosage forms, which comprise a compound of Formula (I) or Formula (II), or a pharmaceutically acceptable salt, solvate, or stereoisomer thereof; and one or more release controlling excipients for use in an enteric coated dosage form. The pharmaceutical compositions may also comprise non-release controlling excipients.

Additionally, provided herein are pharmaceutical compositions in effervescent dosage forms, which comprise the compounds of Formula (I) or Formula (II), or a pharmaceutically acceptable salt, solvate, or stereoisomer thereof; and one or more release controlling excipients for use in an enteric coated dosage form. The pharmaceutical compositions may also comprise non-release controlling excipients.

Further, provided herein are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound of Formula (I) or Formula (II), or a pharmaceutically acceptable salt, solvate, or stereoisomer thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprises a compound of Formula (I) or Formula (II), or a pharmaceutically acceptable salt, solvate, or stereoisomer thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 100 mg, about 0.5 to about 50 mg, about 1 to about 20 mg, about 1 mg, about 2 mg, about 3 mg, about 4 mg, about 5 mg, about 6 mg, about 7 mg, about 8 mg, about 10 mg, about 15 mg, about 20 mg of one or more compounds of Formula (I) or Formula (II) in the form of tablets for oral administration. In some embodiments, the pharmaceutical compositions further comprise hydroxypropyl methylcellulose, lactose, magnesium stearate, micro-crystalline cellulose, polyethylene glycol, sodium starch glycolate, titanium dioxide, and polysorbate 80.

Provided herein are pharmaceutical compositions that comprise about 0.1 to about 100 mg, about 0.5 to about 50 mg, about 1 to about 20 mg, about 1 mg, about 2 mg, about 3 mg, about 4 mg, about 5 mg, about 6 mg, about 7 mg, about 8 mg, about 10 mg, about 15 mg, about 20 mg of one or more compounds of Formula (I) or Formula (II) in the form of coated two-layer tablet for oral administration: one layer that releases a compound of Formula (I) or Formula (II) immediately and another layer that allows a slower release of additional compound of Formula (I) or Formula (II). In some embodiments, the pharmaceutical compositions further comprise colloidal silicon dioxide, hypromellose, lactose monohydrate, magnesium stearate, microcrystalline cellulose, polyethylene glycol, potassium bitartrate, sodium starch glycolate, and titanium dioxide.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

In some embodiments, the compound of Formula (I) or Formula (II) provided herein is administered alone, or in combination with one or more other compounds provided herein, one or more other active ingredients. In some embodiments, the pharmaceutical compositions that comprise a compound provided herein are formulated in various dosage forms for oral, parenteral, and topical administration. In some embodiments, the pharmaceutical compositions are also formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

### A. Oral Administration

The pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmello se; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulo se, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non- effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as other GABA_{A} receptor modulators.

B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

C. Topical Administration

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulo se phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

The pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and ; hydrophilic polymers, such as ethyl cellulose, cellulose acetate, crospovidone, and cross-linked partially hydrolyzed polyvinyl acetate,; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol,; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, β glucan acetate, β glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients.

3. Multiparticulate Controlled Release Devices

The pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

### Cancer

In some aspects, disclosed herein are methods of treating cancer in a subject, comprising administering to a subject a therapeutically effective amount of a compound of Formula (I) or a pharmaceutically acceptable salt, solvate, or stereoisomer thereof:

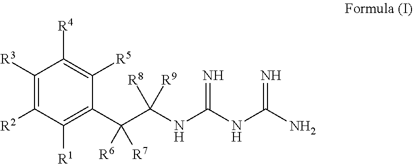

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In some aspect, disclosed herein are methods of treating cancer in a subject, comprising administering to a subject a therapeutically effective amount of a compound of Formula (II) or a pharmaceutically acceptable salt, solvate, or stereoisomer thereof.

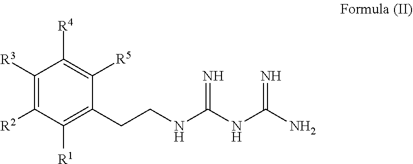

wherein
each of R¹, R², R³, R⁴, and R⁵ is hydrogen or deuterium; wherein at least one R¹, R², R³, R⁴, or R⁵ is deuterium.

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

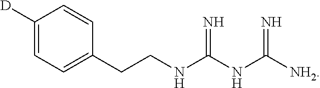 [SMILES: [2H]c1ccc(CCNC(=N)NC(=N)N)cc1]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

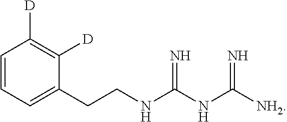 [SMILES: [2H]c1cccc(CCNC(=N)NC(=N)N)c1[2H]]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

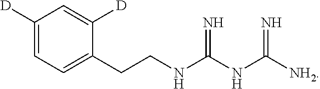 [SMILES: [2H]c1ccc(CCNC(=N)NC(=N)N)c([2H])c1]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

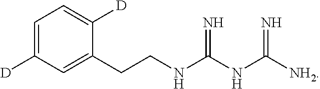 [SMILES: [2H]c1ccc([2H])c(CCNC(=N)NC(=N)N)c1]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

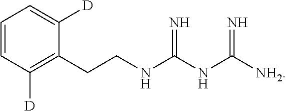 [SMILES: [2H]c1cccc([2H])c1CCNC(=N)NC(=N)N]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

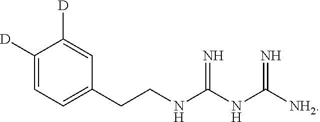 [SMILES: [2H]c1ccc(CCNC(=N)NC(=N)N)cc1[2H]]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

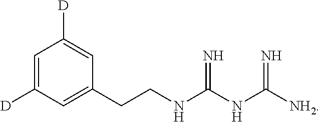 [SMILES: [2H]c1cc([2H])cc(CCNC(=N)NC(=N)N)c1]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

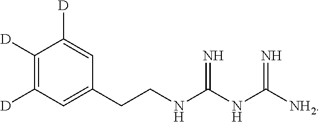 [SMILES: [2H]c1cc(CCNC(=N)NC(=N)N)cc([2H])c1[2H]]

In some embodiments of a method of treating cancer in a subject, comprising administering to a subject a therapeutically effective amount of a compound of Formula (II) or a pharmaceutically acceptable salt, solvate, or stereoisomer thereof, the compound of Formula (II) having the structural formula:

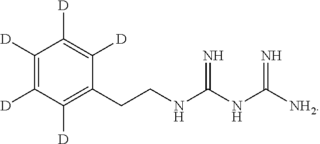 [SMILES: [2H]c1c([2H])c([2H])c(CCNC(=N)NC(=N)N)c([2H])c1[2H]]

In some embodiments of a method of treating cancer in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least one of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In some embodiments of a method of treating cancer in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least two of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In certain embodiments, the average plasma levels of the compound of Formula (I) or Formula (II) are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound of Formula (I) or Formula (II) are decreased by greater than about 5%, greater than about 10%, greater than about 20%, greater than about 30%, greater than about 40%, or greater than about 50% as compared to the corresponding non-isotopically enriched compounds

Plasma levels of the compound of Formula (I) or Formula (II), or metabolites thereof, are measured using the methods described by Li et al. (Rapid Communications in Mass Spectrometry 2005, 19, 1943-1950).

Provided herein are methods for treating cancer, comprising administering to the subject a therapeutically effective amount of a compound of Formula (I) or Formula (II), or a pharmaceutically acceptable salt, solvate, or stereoisomer thereof; so as to affect a decreased metabolism of the compound per dosage unit by at least one polymorphically-expressed cytochrome P450 isoform in the subject, as compared to the corresponding non-isotopically enriched compound.

In some embodiments, the cytochrome P₄₅₀ isoforms in a mammalian subject is selected from CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, the cytochrome P₄₅₀ isoforms in a mammalian subject is CYP2D6.

In certain embodiments, the decrease in metabolism of the compound of Formula (I) or Formula (II) by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

The disclosed methods are useful in the prevention and treatment of solid tumors, soft tissue tumors, and metastases thereof. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

The disclosed methods are useful in the prevention and treatment of LKB1 deficient tumors. In some embodiments, the cancer with LKB1 deficient tumor is a gastrointestinal cancer. In some embodiments, the gastrointestinal cancer is colorectal cancer, small intestinal cancer, gastric cancer, or pancreatic cancer. In some embodiments, the cancer with LKB1 deficient tumor is a gynecological cancer. In some embodiments, the gynecological is breast cancer, ovarian cancer, SCTAT, cervical cancer, prostate cancer, or testicular cancer. In some embodiments, the cancer with LKB1 deficient tumor is a lung cancer. In some embodiments, the lung cancer is non-small cell lung cancer-adenocarcinoma, non-small cell lung cancer-squamous cell, large cell lung cancer, or non-small cell lung cancer. In some embodiments, the cancer with LKB1 deficient tumor is melanoma. In some embodiments, the cancer with LKB1 deficient tumor is soft tissue cancer. In some embodiments, the cancer with LKB1 deficient tumor is renal cancer. In some embodiments, the cancer with LKB1 deficient tumor is brain cancer.

The disclosed methods are also useful in treating non-solid cancers.

Exemplary cancers include, but are not limited to: Adrenocortical Carcinoma, AIDS-Related Cancers (Kaposi Sarcoma, AIDS-Related Lymphoma, Primary CNS Lymphoma), Anal Cancer, Appendix Cancer, Astrocytomas, Atypical Teratoid/Rhabdoid Tumor, Central Nervous System, Basal Cell Carcinoma, Bile Duct Cancer, Bladder Cancer, Bone Cancer Ewing Sarcoma Family of Tumors, Osteosarcoma and Malignant Fibrous Histiocytoma, Brain Stem Glioma, Brain Tumor (Astrocytomas, Central Nervous System Atypical Teratoid/Rhabdoid Tumor, Central Nervous System Embryonal Tumors, Central Nervous System Germ Cell Tumors, Craniopharyngioma, Ependymoma), Breast Cancer, Bronchial Tumors, Carcinoid Tumor, Gastrointestinal, Carcinoma of Unknown Primary, Cardiac (Heart) Tumors, Atypical Teratoid/Rhabdoid Tumor, Embryonal Tumors, Germ Cell Tumor, Cervical Cancer, Cholangiocarcinoma, Chordoma, Chronic Myeloproliferative Neoplasms, Colon Cancer, Colorectal Cancer, Craniopharyngioma, Cutaneous T-Cell Lymphoma , Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Ovarian Cancer, Testicular Cancer, Gestational Trophoblastic Disease, Head and Neck Cancer, Heart Cancer, Hepatocellular (Liver) Cancer, Histiocytosis, Langerhans Cell, Hypopharyngeal Cancer, Intraocular Melanoma, Islet Cell Tumors, Pancreatic Neuroendocrine Tumors, Kidney, Renal Cell, Langerhans Cell Histiocytosis, Laryngeal Cancer, Leukemia (Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia), Lip and Oral Cavity Cancer, Liver Cancer (Primary), Lung Cancer (small cell lung cancer, non-small cell lung cancer), Lymphoma (Burkitt Lymphoma, Hodgkin Lymphoma, Non-Hodgkin Lymphoma, Macroglobulinemia, Waldenström), Male Breast Cancer, Malignant Fibrous Histiocytoma of Bone and Osteosarcoma, Melanoma, Intraocular (Eye), Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myeloma, Multiple Myeloma, Myeloproliferative Neoplasms, Chronic, Nasal Cavity and Paranasal Sinus Cancer, Nasopharyngeal Cancer, Neuroblastoma, Oral Cancer, Lip and Oral Cavity Cancer , Oropharyngeal Cancer, Osteosarcoma and Malignant Fibrous Histiocytoma of Bone, Ovarian Cancer, Epithelial, Germ Cell Tumor, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Osteosarcoma (Bone Cancer), Rhabdomyosarcoma, Soft Tissue, Uterine, Sézary Syndrome, Skin Cancer, Melanoma, Merkel Cell Carcinoma, Non-melanoma Cancer, Small Intestine Cancer, Soft Tissue Sarcoma, Squamous Cell Carcinoma, Squamous Neck Cancer with Occult Primary, Stomach (Gastric) Cancer, Testicular Cancer, Throat Cancer, Thymoma and Thymic Carcinoma, Thyroid Cancer, Transitional Cell Cancer of the Renal Pelvis and Ureter, Ureter and Renal Pelvis, Transitional Cell Cancer, Urethral Cancer, Uterine Cancer, Endometrial cancer, Uterine Sarcoma, Vaginal Cancer, Vulvar Cancer, Wilms Tumor. Metastases of the aforementioned cancers can also be treated or prevented in accordance with the methods described herein.

In some embodiments, the cancer is an IDH1 mutant cancer. In some embodiments, the IDH1 mutant cancer is a glioma, for example, a low grade glioma. In some embodiments, the IDH1 mutant cancer is a sarcoma, for example, chondrosarcoma. In some embodiments, the IDH1 mutant cancer is a carcinoma, for example, intrahepatic cholangiocarcinoma. . In some embodiments, the IDH1 mutant cancer is a leukemia, for example, Acute Myeloid Leukemia (AML). In some embodiments, the IDH1 mutant cancer is a neoplasm, for example Myelodysplastic/Myeloproliferative Neoplasms (MDS/MPN). In some embodiments, the IDH1 mutant cancer is associated with Maffucci syndrome. In some embodiments, the IDH1 mutant cancer is associated with Ollier disease. In some embodiments, the IDH1 mutant cancer is colon cancer, melanoma, lung cancer, or prostate cancer.

### Combination Therapies

In some embodiments, compounds of Formula (I) or Formula (II), or pharmaceutically acceptable salt, solvate, or stereoisomer thereof are administered together with an additional cancer treatment. Exemplary cancer treatments include, for example, chemotherapy, targeted therapies such as antibody therapies, immunotherapy, and hormonal therapy. Examples of each of these treatments are provided below.

### 1. Chemotherapy

In some embodiments, compounds of Formula (I) or Formula (II), or pharmaceutically acceptable salt, solvate, or stereoisomer thereof are administered with chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase. Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Ibrutinib, Idarubicin, Ifosfamide, Irinotecan, Irofulven, Ixabepilone, Larotaxel, Leucovorin, Liposomal doxorubicin, Liposomal daunorubicin. Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein. Because some drugs work better together than alone, two or more drugs are often given at the same time. Often, two or more chemotherapy agents are used as combination chemotherapy.

### 2. Targeted Therapy

In some embodiments, compounds of Formula (I) or Formula (II), or pharmaceutically acceptable salt, solvate, or stereoisomer thereof are administered with a targeted therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as axitinib, bosutinib, cediranib, desatinib, erolotinib, imatinib, gefitinib, lapatinib, lestaurtinib, nilotinib, semaxanib, sorafenib, sunitinib, and vandetanib, and also cyclin-dependent kinase inhibitors such as alvocidib and seliciclib. In some embodiments, the targeted therapy is an IDH1 inhibitor (for example, AGI-5198, AG-120 and AG-881), a Non-Small Cell Lung Cancer SOC agents, an androgen receptor antagonist (for example, bicalutamide, flutamide, nitulamide, apalutamide, enzalutamide, abiraterone acetate, ODM-201, or 4-((1R,2R)-2-Hydroxycyclohexyl)-2(trifluoromethyl)benzonitrile (PF 998425)), or an estrogen receptor antagonist (for example, 7α,17β-[9-[(4,4,5,5,5-pentafluoropentyl)sulfinyl]nonyl]estra-1,3,5(10)-triene-3,17-diol (ICI 182,780), 1,3-Bis(4-hydroxyphenyl)-4-methyl-5-[4-(2-piperidinylethoxy)phenol]-1H-pyrazole dihydrochloride (MPP dihydrochloride), 4-[2-phenyl-5,7-bis(trifluoromethyl)pyrazolo[1,5-a]pyrimidin-3-yl]phenol(PHTPP), 3-[4-(2,4-Bis-trifluoromethylbenzyloxy)-3-methoxyphenyl]-2-cyano-N-(5-trifluoromethyl-1,3,4-thiadiazol-2-yl)acrylamide (XCT 790), or 2-(4-hydroxyphenyl)-3-methyl-1-[10-(pentylsulfonyl)decyl]-1H-indol-5-ol (ZK 164015)). Monoclonal antibody therapy is another strategy in which the therapeutic agent is an antibody which specifically binds to a protein on the surface of the cancer cells. Examples include the anti-HER2/neu antibody trastuzumab typically used in breast cancer, and the anti-CD20 antibody rituximab and tositumomab typically used in a variety of B-cell malignancies. Other exemplary antibodies include ctuximab, panitumumab, trastuzumab, alemtuzumab, bevacizumab, edrecolomab, and gemtuzumab. Exemplary fusion proteins include aflibercept and denileukin diftitox.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell.

### 3. Immunotherapy

In some embodiments, compounds of Formula (I) or Formula (II), or pharmaceutically acceptable salt, solvate, or stereoisomer thereof are administered with an immunotherapy. Cancer immunotherapy refers to a diverse set of therapeutic strategies designed to induce the patient's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma patients.

Allogeneic hematopoietic stem cell transplantation can be considered a form of immunotherapy, since the donor's immune cells will often attack the tumor in a graft-versus-tumor effect.

### 4. Hormonal Therapy

In some embodiments, compounds of Formula (I) or Formula (II), or pharmaceutically acceptable salt, solvate, or stereoisomer thereof are administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

Additional combination therapy.

In some embodiments, compounds of Formula (I) or Formula (II), or pharmaceutically acceptable salt, solvate, or stereoisomer thereof are administered with 2-deoxy glucose, monocarboxylate transporters (for example, MCT1 or MCT4), or glucose transporters (for example GLUT4).

### Diabetes

In another aspect, disclosed herein are methods of treating diabetes in a subject, comprising administering to a subject a therapeutically effective amount of a compound of Formula (I) or a pharmaceutically acceptable salt, solvate, or stereoisomer thereof:

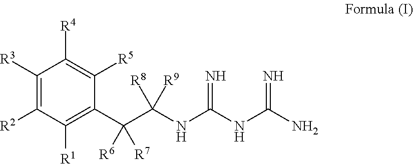

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In another aspect, disclosed herein are methods of treating diabetes in a subject, comprising administering to a subject a therapeutically effective amount of a compound of Formula (II) or a pharmaceutically acceptable salt, solvate, or stereoisomer thereof.

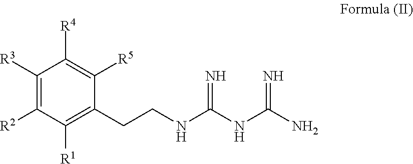

wherein
each of R¹, R², R³, R⁴, and R⁵ is hydrogen or deuterium; wherein at least one R¹, R², R³, R⁴, or R⁵ is deuterium.

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

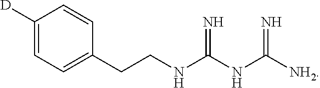 [SMILES: [2H]c1ccc(CCNC(=N)NC(=N)N)cc1]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

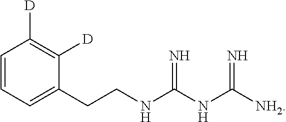 [SMILES: [2H]c1cccc(CCNC(=N)NC(=N)N)c1[2H]]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

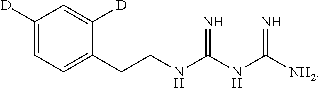 [SMILES: [2H]c1ccc(CCNC(=N)NC(=N)N)c([2H])c1]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

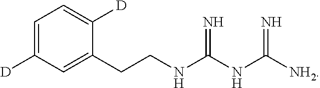 [SMILES: [2H]c1ccc([2H])c(CCNC(=N)NC(=N)N)c1]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

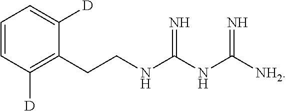 [SMILES: [2H]c1cccc([2H])c1CCNC(=N)NC(=N)N]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

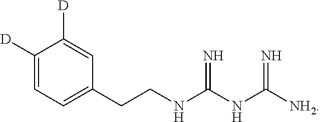 [SMILES: [2H]c1ccc(CCNC(=N)NC(=N)N)cc1[2H]]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

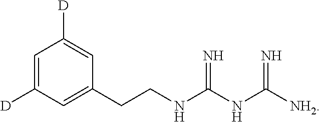 [SMILES: [2H]c1cc([2H])cc(CCNC(=N)NC(=N)N)c1]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

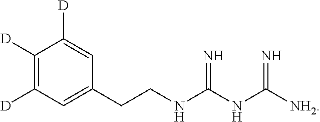 [SMILES: [2H]c1cc(CCNC(=N)NC(=N)N)cc([2H])c1[2H]]

In some embodiments of a method of treating diabetes in a subject, comprising administering to a subject a therapeutically effective amount of a compound of Formula (II) or a pharmaceutically acceptable salt, solvate, or stereoisomer thereof, the compound of Formula (II) having the structural formula:

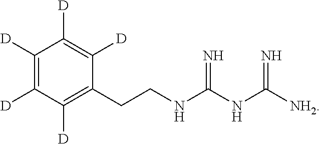 [SMILES: [2H]c1c([2H])c([2H])c(CCNC(=N)NC(=N)N)c([2H])c1[2H]]

In some embodiments of a method of treating diabetes in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least one of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In some embodiments of a method of treating diabetes in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least two of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In certain embodiments, the average plasma levels of the compound of Formula (I) or Formula (II) are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound of Formula (I) or Formula (II) are decreased by greater than about 5%, greater than about 10%, greater than about 20%, greater than about 30%, greater than about 40%, or greater than about 50% as compared to the corresponding non-isotopically enriched compounds

Plasma levels of the compound of Formula (I) or Formula (II), or metabolites thereof, are measured using the methods described by Li et al. (Rapid Communications in Mass Spectrometry 2005, 19, 1943-1950).

Provided herein are methods for treating diabetes, comprising administering to the subject a therapeutically effective amount of a compound of Formula (I) or Formula (II), or a pharmaceutically acceptable salt, solvate, or stereoisomer thereof; so as to affect a decreased metabolism of the compound per dosage unit by at least one polymorphically-expressed cytochrome P450 isoform in the subject, as compared to the corresponding non-isotopically enriched compound.

In some embodiments, the cytochrome P₄₅₀ isoforms in a mammalian subject is selected from CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, the cytochrome P₄₅₀ isoforms in a mammalian subject is CYP2D6.

In certain embodiments, the decrease in metabolism of the compound of Formula (I) or Formula (II) by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

Methods disclose herein are used for the treatment and/or prevention of diabetes, including, for example, type 1 and type 2 diabetes, and related diseases. In some embodiments, the methods are used for treating prediabetes, and/or glucose intolerance. In some embodiments the methods help with glycemic control, as monitored by, for example, average glucose and/or glycosylated hemoglobin levels. Further, in some embodiments, the patient being treated may suffer from insulin resistance. In other aspects, the present invention provides for treatments and uses in the prevention of diabetes onset in patients afflicted with, for example, insulin resistance, prediabetes, impaired fasting glucose (IFG), impaired glucose tolerance (IGT), and acanthosis nigricans.

### Combination Therapies

In some embodiments, compounds of Formula (I) or Formula (II), or pharmaceutically acceptable salt, solvate, or stereoisomer thereof are administered together with an additional diabetes treatment. Exemplary diabetes treatments include:

Insulin and insulin derivatives are selected from insulin glargine, insulin glulisine, insulin detemir, insulin lispro, insulin degludec, insulin aspart, basal insulin and analogues (e.g. LY2605541, LY2963016, NN1436), PEGylated insulin lispro (e.g. LY-275585), long-acting insulins, intermediate-acting insulins, and fast-acting and short-acting insulins. Also suitable are those insulin derivatives which are bonded to albumin or another protein by a bifunctional linker.

Glucagon-like-peptide 1 (GLP-1), GLP-1 analogues, and GLP-1 receptor agonists, for example: lixisenatide, exenatide, liraglutide, semaglutide, taspoglutide, albiglutide, dulaglutide, ACP-003, CJC-1 134-PC, GSK-2374697, PB-1023, TTP-054, langlenatide (HM-1 1260C), CM-3, GLP-1 Eligen, AB-201, ORMD-0901, NN9924, NN9926, NN9927, Nodexen, Viador-GLP-1, CVX-096, ZYOG-1, ZYD-1, ZP-3022, CAM- 2036, DA-3091, DA-15864, ARI-2651, ARI-2255, exenatide-XTEN (VRS-859), exenatide-XTEN+ Glucagon-XTEN (VRS-859+AMX-808) and polymer-bound GLP-1 and GLP-1 analogues. Dual GLP-1/GIP agonists (e.g. RG-7697 (MAR-701), MAR-709, BHM081, BHM089, BHM098). Dual GLP-1/glucagon receptor agonists (e.g. BHM-034, OAP-189 (PF-05212389, TKS- 1225), TT-401/402, ZP2929, LAPS-HMOXM25, MOD-6030). Dual GLP-1 /gastrin agonists (e.g. ZP-3022).

Glucagon receptor agonists or antagonists, glucose-dependent insulinotropic polypeptide (GIP) receptor agonists or antagonists, ghrelin antagonists or inverse agonists, xenin and analogues thereof, dipeptidyl peptidase-IV (DPP-4) inhibitors, for example: alogliptin, linagliptin, saxagliptin, sitagliptin, anagliptin, teneligliptin, trelagliptin, vildagliptin, gemigliptin, omarigliptin, evogliptin, dutogliptin, DA-1229, MK-3102, KM-223, KRP-104, PBL-1427, Pinoxacin hydrochloride, and Ari-2243, sodium-dependent glucose transporter 2 (SGLT-2) inhibitors, for example: canagliflozin, dapagliflozin, remogliflozin, remogliflozin etabonate, sergliflozin, empagliflozin, ipragliflozin, tofogliflozin, luseogliflozin, ertugliflozin, EGT-0001442, LIK-066, SBM-TFC- 039, and KGA-3235 (DSP-3235), dual inhibitors of SGLT-2 and SGLT-1 (e.g. LX-421 1, LIK066), biguanides (e.g. metformin, buformin), thiazolidinediones (e.g. pioglitazone, rosiglitazone), glitazone analogues (e.g. lobeglitazone), peroxisome proliferator-activated receptors (PPAR-)(alpha, gamma or alpha/gamma) agonists or modulators (e.g. saroglitazar), or PPAR gamma partial agonists (e.g. lnt-131).

Sulfonylureas (e.g. tolbutamide, glibenclamide, glimepiride, glipizide) and meglitinides (e.g. nateglinide, repaglinide, mitiglinide), alpha-glucosidase inhibitors (e.g. acarbose, miglitol, voglibose), amylin and amylin analogues (e.g. pramlintide) G-protein coupled receptor 1 19 (GPR1 19) agonists (e.g. GSK-1292263, PSN-821, MBX-2982, APD-597, ARRY-981, ZYG-19, DS-8500, HM-47000, YH-Cheml), GPR40 agonists (e.g. TUG-424, P-1736, P-1 1 187, JTT-851, GW9508, CNX-01 1-67, AM-1638, AM-5262), GPR120 agonists and GPR142 agonists, systemic or low-absorbable TGRS (GPBAR1=G-protein-coupled bile acid receptor 1) agonists (e.g. INT-777, XL-475, SB756050).

Diabetes immunotherapeutics, for example: oral C-C chemokine receptor type 2 (CCR- 2) antagonists (e.g. CCX-140, JNJ-41443532), interleukin 1 beta (IL-1 .beta.) antagonists (e.g. AC-201), or oral monoclonal antibodies (MoA) (e.g. methalozamide, WP808, PAZ-320, P-1736, PF-05175157, PF-04937319).

Anti-inflammatory agents for the treatment of the metabolic syndrome and diabetes, for example: nuclear factor kappa B inhibitors. Adenosine monophosphate-activated protein kinase (AMPK) stimulants, for example: Imeglimin (PXL-008), Debio-0930 (MT-63-78), R-1 18. Inhibitors of 11-beta-hydroxysteroid dehydrogenase 1 (11-beta-HSD-1) (e.g. LY2523199, BMS770767, RG-4929, BMS816336, AZD-8329, HSD-016, BI-135585).

Activators of glucokinase (e.g. PF-04991532, TTP-399 (GK1-399), GKM-001 (ADV-1002401), ARRY-403 (AMG-151), TAK-329, TMG-123, ZYGK1).

Inhibitors of diacylglycero10-acyltransferase (DGAT) (e.g. pradigastat (LCQ-908)), inhibitors of protein tyrosine phosphatase 1 (e.g. trodusquemine), inhibitors of glucose- 6-phosphatase, inhibitors of fructose-1 ,6-bisphosphatase, inhibitors of glycogen phosphorylase, inhibitors of phosphoenol pyruvate carboxykinase, inhibitors of glycogen synthase kinase, inhibitors of pyruvate dehydrogenase kinase.

Modulators of glucose transporter-4, somatostatin receptor 3 agonists (e.g. MK-4256).

One or more lipid lowering agents, for example: 3-hydroxy-3-methylglutaryl-coenzym-A-reductase (HMG-CoA-reductase) inhibitors such as simvastatin, atorvastatin, rosuvastatin, pravastatin, fluvastatin, pitavastatin, lovastatin, mevastatin, rivastatin, cerivastatin, fibrates such as bezafibrate, ciprofibrate, fenofibrate, gemfibrozil, etofibrate, simfibrate, ronifibrate, clinofibrate, clofibride, nicotinic acid and derivatives thereof (e.g. niacin, including slow release formulations of niacin), nicotinic acid receptor 1 agonists (e.g. GSK-256073), PPAR-delta agonists, acetyl-CoA-acetyltransferase (ACAT) inhibitors (e.g. avasimibe), cholesterol absorption inhibitors (e.g. ezetimibe, S-556971), bile acid-binding substances (e.g. cholestyramine, colesevelam), ileal bile acid transport (IBAT) inhibitors (e.g. GSK-2330672, LUM-002), microsomal triglyceride transfer protein (MTP) inhibitors (e.g. lomitapide (AEGR-733), SLx-4090, granotapide), modulators of proprotein convertase subtilisin/kexin type 9 (PCSK9) (e.g. alirocumab (REGN727/SAR236553), AMG-145, LGT-209, PF-04950615, MPSK3169A, LY3015014, ALD-306, ALN-PCS, BMS-962476, SPC5001, ISIS-394814, 1 B20, LGT-210, 1 D05, BMS-PCSK9Rx-2, SX-PCK9, RG7652), LDL receptor up- regulators, for example liver selective thyroid hormone receptor beta agonists (e.g. eprotirome (KB-21 15), MB0781 1, sobetirome (QRX-431), VIA-3196, ZYT1), HDL-raising compounds such as: cholesteryl ester transfer protein (CETP) inhibitors (e.g. anacetrapib (MK0859), dalcetrapib, evacetrapib, JTT-302, DRL-17822, TA-8995, R- 1658, LY-2484595, DS-1442), or dual CETP/PCSK9 inhibitors (e.g. K-312), ATP-binding cassette (ABC1) regulators, lipid metabolism modulators (e.g. BMS-823778, TAP-301, DRL-21994, DRL-21995), phospholipase A2 (PLA2) inhibitors (e.g. darapladib, varespladib, rilapladib), ApoA-1 enhancers (e.g. RVX-208, CER-001, MDCO-216, CSL-1 12), cholesterol synthesis inhibitors (e.g. ETC-1002), lipid metabolism modulators (e.g. BMS-823778, TAP-301, DRL-21994, DRL-21995) and omega-3 fatty acids and derivatives thereof (e.g. icosapent ethyl (AMR101), AKR-063, NKPL-66, PRC-4016, CAT-2003).

Treatment of obesity, such as for example:

Bromocriptine, phentermine and phentermine formulations or combinations (e.g. Adipex-P, lonamin), benzphetamine, diethylpropion, phendimetrazin, bupropion and combinations, sibutramine, topiramat, zonisamid, tesofensine, opioid antagonists such as naltrexone, cannabinoid receptor 1 (CB1) antagonists (e.g. TM-38837), melanin- concentrating hormone (MCH-1) antagonists (e.g. BMS-830216, ALB-127158(a)), MC4 receptor agonists and partial agonists (e.g. AZD-2820, RM-493), neuropeptide Y5 (NPY5) or NPY2 antagonists (e.g. velneperit, S-234462), NPY4 agonists (e.g. PP-1420), beta-3-adrenergic receptor agonists, leptin or leptin mimetics, agonists of the 5- hydroxytryptamine 2c (5HT2c) receptor (e.g. lorcaserin), pramlintide/metreleptin, lipase inhibitors such as cetilistat, orlistat, angiogenesis inhibitors (e.g. ALS-L1023), betahistidin and histamine H3 antagonists (e.g. HPP-404), AgRP (agouti related protein) inhibitors (e.g. TTP-435), serotonin re-uptake inhibitors such as fluoxetine, duloxetine, dual or triple monoamine uptake inhibitors (dopamine, norepinephrine and serotonin re-uptake) such as sertraline, tesofensine, methionine aminopeptidase 2 (MetAP2) inhibitors (e.g. beloranib), and antisense oligonucleotides against production of fibroblast growth factor receptor 4 (FGFR4) (e.g. ISIS-FGFR4Rx) or prohibitin targeting peptide-1.

Drugs for influencing high blood pressure, chronic heart failure or atherosclerosis, for example: nitric oxide donors, AT1 antagonists or angiotensin II (AT2) receptor antagonists such as telmisartan, candesartan, valsartan, losartan, eprosartan, irbesartan, olmesartan, tasosartan, azilsartan, dual angiotensin receptor blockers (dual ARBs), angiotensin converting enzyme (ACE) inhibitors, ACE-2 activators, renin inhibitors, prorenin inhibitors, endothelin converting enzyme (ECE) inhibitors, endothelin receptor (ET1/ETA) blockers, endothelin antagonists, diuretics, aldosterone antagonists, aldosterone synthase inhibitors, alpha- blockers, antagonists of the alpha-2 adrenergic receptor, beta-blockers, mixed alpha-/beta-blockers, calcium antagonists, calcium channel blockers (CCBs), nasal formulations of the calcium channel blocker diltiazem (e.g. CP-404), dual

mineralocorticoid/CCB s, centrally acting antihypertensives, inhibitors of neutral endopeptidase, aminopeptidase-A inhibitors, vasopeptide inhibitors, dual vasopeptide inhibitors such as neprilysin-ACE inhibitors or neprilysin-ECE inhibitors, dual-acting AT receptor-neprilysin inhibitors, dual AT1/ETA antagonists, advanced glycation end- product (AGE) breakers, recombinant renalase, blood pressure vaccines such as anti- RAAS (renin-angiotensin-aldosteron-system) vaccines, AT1- or AT2-vaccines, drugs based on hypertension pharmacogenomics such as modulators of genetic.

### Additional Combination Therapy.

In some embodiments, compounds of Formula (I) or Formula (II), or pharmaceutically acceptable salt, solvate, or stereoisomer thereof are administered with 2-deoxy glucose, monocarboxylate transporters (for example, MCT1 or MCT4), or glucose transporters (for example GLUT4).

Depending on the disease to be treated and the subject's condition, the compound of Formula (I) or Formula (II) provided herein may be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1000 milligram, from about 0.1 to about 500 milligrams, or from 0.5 about to about 100 milligram active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Polycystic Ovarian Syndrome (PCOS)

In another aspect, disclosed herein are methods of treating polycystic ovarian syndrome (PCOS) in a subject, comprising administering to a subject a therapeutically effective amount of a compound of Formula (I) or a pharmaceutically acceptable salt, solvate, or stereoisomer thereof:

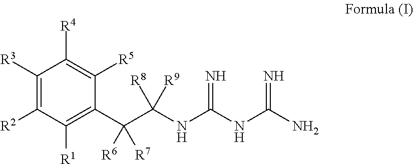

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

In another aspect, disclosed herein are methods of treating polycystic ovarian syndrome (PCOS) in a subject, comprising administering to a subject a therapeutically effective amount of a compound of Formula (II) or a pharmaceutically acceptable salt, solvate, or stereoisomer thereof.

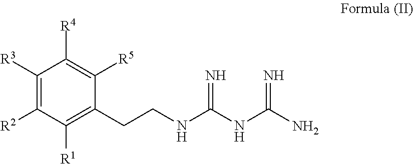

wherein
each of R¹, R², R³, R⁴, and R⁵ is hydrogen or deuterium; wherein at least one R¹, R², R³, R⁴, or R⁵ is deuterium.

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

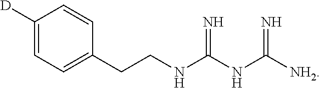 [SMILES: [2H]c1ccc(CCNC(=N)NC(=N)N)cc1]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

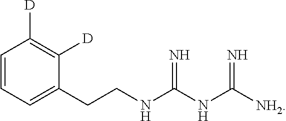 [SMILES: [2H]c1cccc(CCNC(=N)NC(=N)N)c1[2H]]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

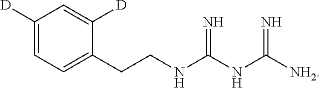 [SMILES: [2H]c1ccc(CCNC(=N)NC(=N)N)c([2H])c1]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

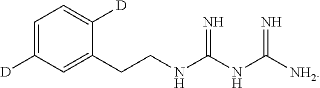 [SMILES: [2H]c1ccc([2H])c(CCNC(=N)NC(=N)N)c1]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

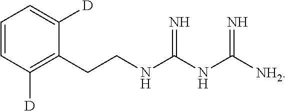 [SMILES: [2H]c1cccc([2H])c1CCNC(=N)NC(=N)N]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

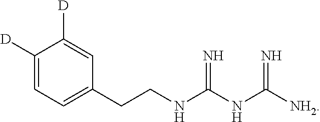 [SMILES: [2H]c1ccc(CCNC(=N)NC(=N)N)cc1[2H]]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

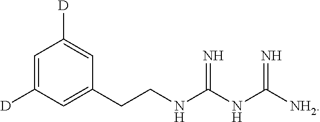 [SMILES: [2H]c1cc([2H])cc(CCNC(=N)NC(=N)N)c1]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

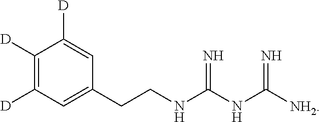 [SMILES: [2H]c1cc(CCNC(=N)NC(=N)N)cc([2H])c1[2H]]

In some embodiments of a method of treating polycystic ovarian syndrome (PCOS) in a subject, comprising administering to a subject a therapeutically effective amount of a compound of Formula (II) or a pharmaceutically acceptable salt, solvate, or stereoisomer thereof, the compound of Formula (II) having the structural formula:

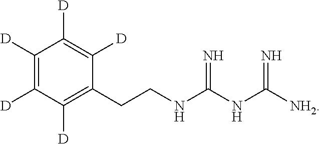 [SMILES: [2H]c1c([2H])c([2H])c(CCNC(=N)NC(=N)N)c([2H])c1[2H]]

In some embodiments of a method of treating polycystic ovarian syndrome (PCOS) in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least one of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In some embodiments of a method of treating polycystic ovarian syndrome (PCOS) in a subject, comprising administering to a subject a therapeutically effective amount of a compound of Formula (I) or Formula (II) or a pharmaceutically acceptable salt, solvate, or stereoisomer thereof, the compound of Formula (I) or Formula (II) has at least two of the following properties:
a) decreased inter-individual variation in plasma levels of the compound or a metabolite thereof as compared to the non-isotopically enriched compound;
b) increased average plasma levels of the compound per dosage unit as compared to the non-isotopically enriched compound;
c) decreased average plasma levels of at least one metabolite of the compound per dosage unit as compared to the non-isotopically enriched compound;
d) improved clinical effect during the treatment in the subject per dosage unit as compared to the non-isotopically enriched compound.

In certain embodiments, the average plasma levels of the compound of Formula (I) or Formula (II) are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound of Formula (I) or Formula (II) are decreased by greater than about 5%, greater than about 10%, greater than about 20%, greater than about 30%, greater than about 40%, or greater than about 50% as compared to the corresponding non-isotopically enriched compounds

Plasma levels of the compound of Formula (I) or Formula (II), or metabolites thereof, are measured using the methods described by Li et al. (Rapid Communications in Mass Spectrometry 2005, 19, 1943-1950).

Provided herein are methods for treating polycystic ovarian syndrome (PCOS), comprising administering to the subject a therapeutically effective amount of a compound of Formula (I) or Formula (II), or a pharmaceutically acceptable salt, solvate, or stereoisomer thereof; so as to affect a decreased metabolism of the compound per dosage unit by at least one polymorphically-expressed cytochrome P450 isoform in the subject, as compared to the corresponding non-isotopically enriched compound.

In some embodiments, the cytochrome P₄₅₀ isoforms in a mammalian subject is selected from CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, the cytochrome P₄₅₀ isoforms in a mammalian subject is CYP2D6.

In certain embodiments, the decrease in metabolism of the compound of Formula (I) or Formula (II) by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

### Combination Therapies

In some embodiments, compounds of Formula (I) or Formula (II), or pharmaceutically acceptable salt, solvate, or stereoisomer thereof are administered together with an additional polycystic ovarian syndrome (PCOS) treatment. Exemplary treatments include: metformin and oral contraceptive.

Combination Therapies with CYP Inhibitor

Provided herein are methods of increasing the bioavailability of a compound of Formula (I) in a subject comprising administering to a subject a compound of Formula (I), or a pharmaceutically acceptable salt, solvate, or stereoisomer thereof:

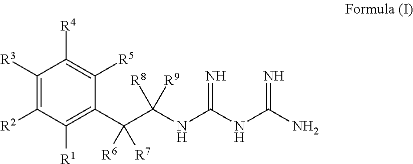

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium;
and a cytochrome P450 inhibitor.

Provided herein are methods of increasing the bioavailability of a compound of Formula (II) in a subject comprising administering to a subject a compound of Formula (II), or a pharmaceutically acceptable salt, solvate, or stereoisomer thereof:

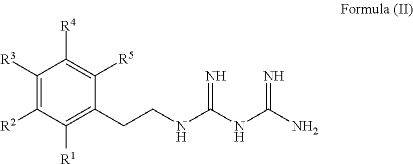

wherein
each of R¹, R², R³, R⁴, and R⁵ is hydrogen or deuterium; wherein at least one R¹, R², R³, R⁴, or R⁵ is deuterium;
and a cytochrome P450 inhibitor.

Provided herein are methods of increasing the bioavailability of a compound of Formula (II) in a subject comprising administering to a subject a compound of Formula (II), or a pharmaceutically acceptable salt, solvate, or stereoisomer thereof, the compound of Formula (II) having the structural formula:

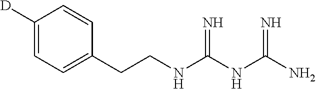 [SMILES: [2H]c1ccc(CCNC(=N)NC(=N)N)cc1]

and a cytochrome P450 inhibitor.

Provided herein are methods of increasing the bioavailability of a compound of Formula (II) in a subject comprising administering to a subject a compound of Formula (II), or a pharmaceutically acceptable salt, solvate, or stereoisomer thereof, the compound of Formula (II) having the structural formula:

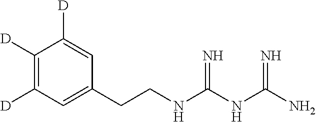 [SMILES: [2H]c1cc(CCNC(=N)NC(=N)N)cc([2H])c1[2H]]

and a cytochrome P450 inhibitor.

Provided herein are methods of increasing the bioavailability of a compound of Formula (II) in a subject comprising administering to a subject a compound of Formula (II), or a pharmaceutically acceptable salt, solvate, or stereoisomer thereof, the compound of Formula (II) having the structural formula:

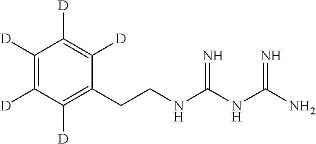 [SMILES: [2H]c1c([2H])c([2H])c(CCNC(=N)NC(=N)N)c([2H])c1[2H]]

and a cytochrome P450 inhibitor.

Provided herein are methods of decreasing the metabolism of a compound of Formula (I) in a subject comprising administering to a subject a compound of Formula (I), or a pharmaceutically acceptable salt, solvate, or stereoisomer thereof:

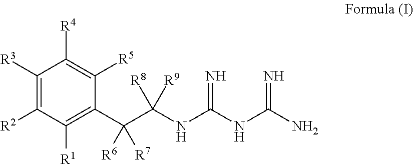

wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium;
and a cytochrome P450 inhibitor.

Provided herein are methods of decreasing the metabolism of a compound of Formula (II) in a subject comprising administering to a subject a compound of Formula (II), or a pharmaceutically acceptable salt, solvate, or stereoisomer thereof:

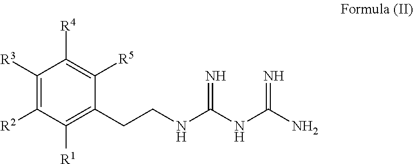

wherein
each of R¹, R², R³, R⁴, and R⁵ is hydrogen or deuterium; wherein at least one R¹, R², R³, R⁴, or R⁵ is deuterium;
and a cytochrome P450 inhibitor.

Provided herein are methods of decreasing the metabolism of a compound of Formula (II) in a subject comprising administering to a subject a compound of Formula (II), or a pharmaceutically acceptable salt, solvate, or stereoisomer thereof, the compound of Formula (II) having the structural formula:

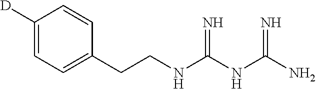 [SMILES: [2H]c1ccc(CCNC(=N)NC(=N)N)cc1]

and a cytochrome P450 inhibitor.

Provided herein are methods of decreasing the metabolism of a compound of Formula (II) in a subject comprising administering to a subject a compound of Formula (II), or a pharmaceutically acceptable salt, solvate, or stereoisomer thereof, the compound of Formula (II) having the structural formula:

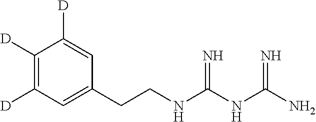 [SMILES: [2H]c1cc(CCNC(=N)NC(=N)N)cc([2H])c1[2H]]

and a cytochrome P450 inhibitor.

Provided herein are methods of decreasing the metabolism of a compound of Formula (II) in a subject comprising administering to a subject a compound of Formula (II), or a pharmaceutically acceptable salt, solvate, or stereoisomer thereof, the compound of Formula (II) having the structural formula:

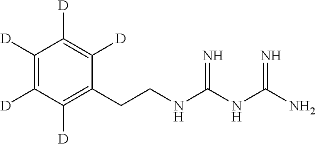 [SMILES: [2H]c1c([2H])c([2H])c(CCNC(=N)NC(=N)N)c([2H])c1[2H]]

and a cytochrome P450 inhibitor.

Provided herein are methods of increasing the bioavailability of phenformin in a subject comprising administering to a subject phenformin, or a pharmaceutically acceptable salt, solvate, or stereoisomer thereof and a cytochrome P450 inhibitor.

Provided herein are methods of decreasing the metabolism of a compound of phenformin in a subject comprising administering to a subject phenformin, or a pharmaceutically acceptable salt, solvate, or stereoisomer thereof and a cytochrome P450 inhibitor.

In some embodiments, the cytochrome P450 inhibitor is a cytochrome P450 2D6 inhibitor. In some embodiments, the cytochrome P450 2D6 inhibitor is selected from amiodarone, buprenorphine, bupropion, cannabidiol, celecoxib, chlorphenamine, chlorpromazine, cimetidine, cinacalcet, citalopram, clemastine, clomipramine, diphenhydramine, doxepin, doxorubicin, duloxetine, escitalopram, fluoxetine, halofantrine, haloperidol, hydroxyzine, hyperforin, levomepromazine, methadone, metoclopramide, mibefradil, midodrine, moclobemide, paroxetine, perphenazine, promethazine, quinidine, risperidone, ritonavir, sertraline, terbinafine, thioridazine, ticlopidine, tripelennamine, and zuclopenthixol. In some embodiments, the cytochrome P450 2D6 inhibitor is quinidine.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds of Formula (I) or Formula (II), optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

### EXAMPLES

For all of the following examples, standard work-up and purification methods known to those skilled in the art can be utilized. Synthetic methodologies illustrated in Schemes 2, 3, and 4 is intended to exemplify the applicable chemistry through the use of specific examples and are not indicative of the scope of what is claimed herein.

### Example 1

### Synthesis of Compound 1

The synthesis of compound 1 is done according to scheme 2. Deuterated 3-phenylpropanenitrile (1-a) is reduced to afford deuterated 2-phenylethanamine (1-b) which is then reacted with cyanated guanidine derivative (1-c) to afford compound 1 (as described in Journal of the American Chemical Society, 81, 2220-2225; 1959).

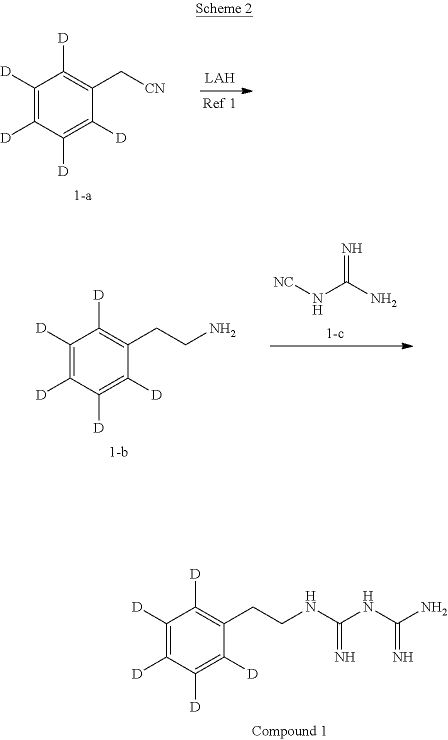 [SMILES: [2H]c1c([2H])c([2H])c(CC#N)c([2H])c1[2H] ; [2H]c1c([2H])c([2H])c(CCN)c([2H])c1[2H] ; N#CNC(=N)N ; [2H]c1c([2H])c([2H])c(CCNC(=N)NC(=N)N)c([2H])c1[2H]]

### Example 2

### Synthesis of Compound 2

The synthesis of compound 2 is done according to scheme 3. 1,4-Dibromobenzene (2-a) is reacted with Mg in the presence of D₂O to form mono-deuterated bromobenzene (2-b) (as described in Journal of Organic Chemistry, 48(20), 3458-3464, 1983) which is further reacted with oxirane to afford mono-deuterated 2-phenylethanol (2-c) (as described in Journal of Labelled Compounds and Radiopharmaceuticals, 48(12), 897-907; 2005). Intermediate 2-c is then converted to mono-deuterated 2-phenylethanamine (2-f) via mono-deuterated intermediates 2-d and 2-e. Mono-deuterated 2-phenylethanamine (2-f) is then reacted with cyanated guanidine derivative (2-g) to afford compound 2 (as described in Journal of the American Chemical Society, 81, 2220-2225; 1959).

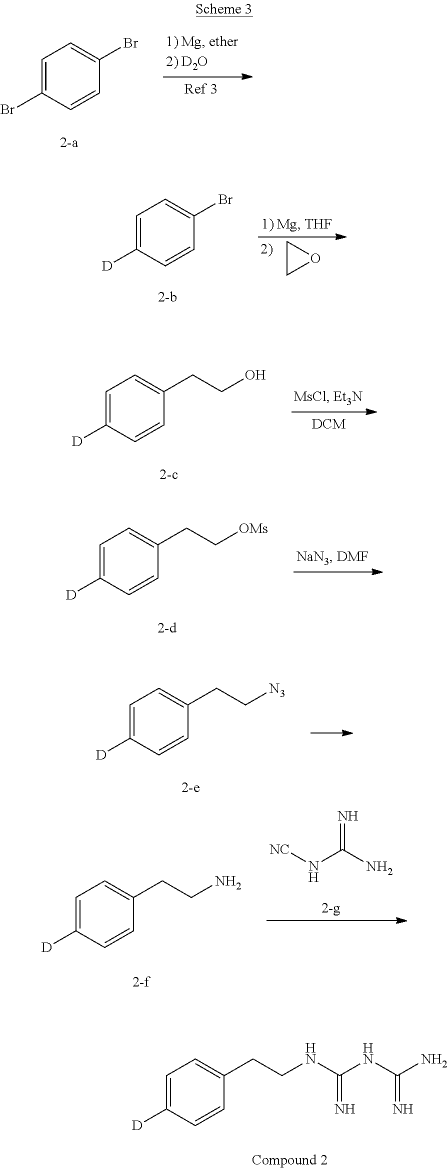

### Example 3

### Synthesis of Compound 3

The synthesis of compound 3 is done according to scheme 4. 4-Bromoaniline (3-a) is converted to tri-deuterated bromobenzene (3-b) (as described in Journal of Chemical and Engineering Data, 55(5), 2048-2054; 2010) which is further reacted with oxirane to afford tri-deuterated 2-phenylethanol (3-c) (as described in Journal of Labelled Compounds and Radiopharmaceuticals, 48(12), 897-907; 2005). Intermediate 3-c is then converted to tri-deuterated 2-phenylethanamine (3-f) via tri-deuterated intermediates 3-d and 3-e. tri-deuterated 2-phenylethanamine (3-f) is then reacted with cyanated guanidine derivative (3-g) to afford compound 3 (as described in Journal of the American Chemical Society, 81, 2220-2225; 1959).

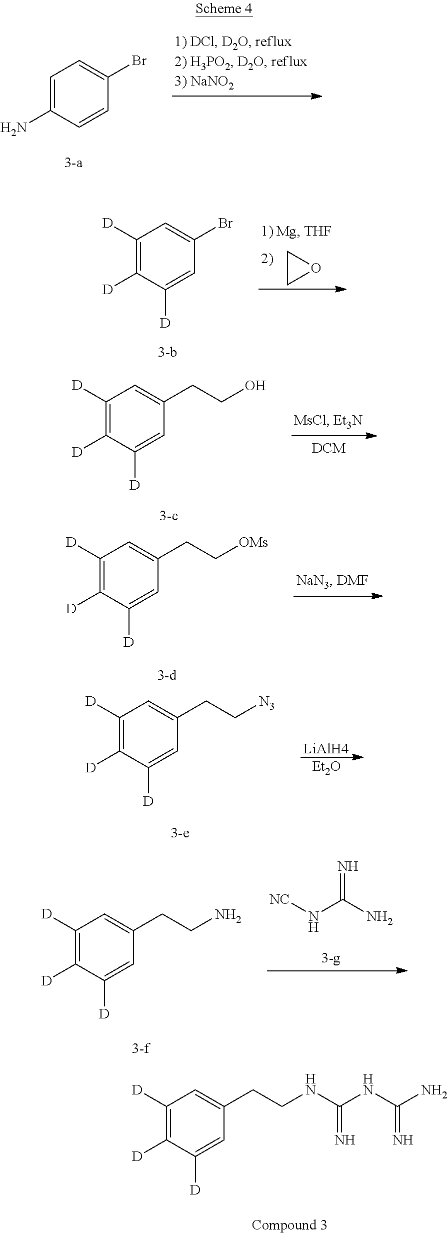

### Example 4

### Evaluation of Clinical Safety of Combining Compound 2 with Anticancer Chemotherapy (Phase 1)

Study Interventional
Type:

Study Allocation: Randomized
Design: Endpoint Classification: Safety Study
   Intervention Model: Parallel Assignment
   Masking: Open Label
   Primary Purpose: Treatment

Primary Outcome Measures:
Incidence of dose limiting toxicity when Compound 2 is added to chemotherapy [Time Frame: 1 cycle (at least 3 weeks)] [Designated as safety issue: Yes]

The primary endpoint of the study will be to determine whether Compound 2 can be safely added to a chemotherapy regimen that is previously well tolerated. The rate of dose limiting toxicities will be compared.

Secondary Outcome Measures:
Number of Participants with Adverse Events as a Measure of Safety and Tolerability [Time Frame: 1 cycle (at least 3 weeks) ] [ Designated as safety issue: Yes ]

Secondary endpoints will include assessment of AEs≧grade 3 and Serious Adverse Events (SAEs), assessment of safety beyond the first cycle with Compound 2, and an exploration of Compound 2-chemotherapy drug interactions.

| | Assigned |
|---|---|
| Arms | Interventions |
| Experimental: Compound 2 | Drug: |
| | Compound 2 |
| No Intervention: No Compound 2 | |
| No Compound 2 during primary endpoint assessment | |
| period (at least 3 weeks). Patients will subsequently | |
| be initiated on Compound 2. | |

Eligibility
Ages Eligible for Study: 18 Years or older
Genders Eligible for Study: Both
Accepts healthy Volunteers: No
Criteria

Inclusion Criteria:
Histologically or cytologically documented cancer; diagnosis of hepatocellular carcinoma may be made by characteristic radiographic and/or AFP findings 33;
Intended treatment with, or currently being treated by anti-cancer chemotherapy in the adjuvant or advanced setting;
Age 18 to 79;
Adequate renal function (serum creatinine levels<1.5 mg/dL [males], <1.4 mg/dL [females]). If a subject does not meet these criteria, but does have an estimated creatinine clearance>=60 ml/min using the Cockroft-Gault calculation, they will be allowed. The Cockroft-Gault formula is CrCl=(140-age)× weight(kg)÷(Cr×72), where CrCl=estimated creatinine clearance and Cr is plasma creatinine in mg/dL;
Adequate hepatic parameters, including aspartate aminotransferase (AST) and alanine aminotransferase (ALT) levels<2.5× upper limit of normal (ULN), total bilirubin≦1.5× ULN, and alkaline phosphatase levels≦2.5× ULN;
Must anticipate receiving at least 3 cycles (or treatment periods of at least 3-weeks) of chemotherapy;
Ability to understand and willingness to sign a written informed consent document.

Exclusion Criteria:
Current use of metformin (within 1 week of start of chemotherapy regimen to be assessed);
Patients with type 2 diabetes are allowed, however they will be excluded if there is intent to use metformin for treatment of diabetes during the course of the study;
Undergoing chemotherapy treatment concurrent with radiation therapy;
Undergoing chemotherapy in a neoadjuvant setting prior to potentially curative surgery;
Renal disease or renal dysfunction not meeting inclusion criteria;
Significant medical conditions such as cardiovascular collapse (shock), acute myocardial infarction, septicemia, acute or chronic metabolic acidosis;
History of, or states associated with, lactic acidosis such as shock or pulmonary insufficiency, alcoholism (acute or chronic), conditions associated with hypoxemia and pancreatitis;
Severe dehydration;
Clinical or laboratory evidence of hepatic disease;
Congestive heart failure requiring pharmacologic treatment, or unstable or acute congestive heart failure;
Known hypersensitivity to a biguanide compounds;
Pregnant or lactating women (serum pregnancy test will be performed for all women of child-bearing potential);
Psychiatric illness or social situation that would limit compliance with study requirements and/or obscure results

What is claimed is:

1. A compound of Formula (II) or a pharmaceutically acceptable salt, solvate, or stereoisomer thereof, 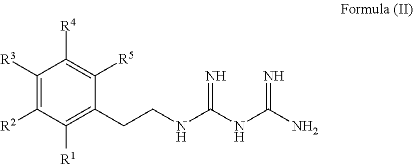
wherein
each of R¹, R², R³, R⁴, and R⁵ is hydrogen or deuterium; wherein at least one R¹, R², R³, R⁴, or R⁵ is deuterium.

2. The compound of claim 1, wherein R³ is deuterium; and R¹, R², R⁴, and R⁵ are each hydrogen.

3. The compound of claim 1, wherein R¹, R², R³, R⁴, and R⁵ are all deuterium.

4. The compound of claim 1, wherein at least two of R¹, R², R³, R⁴, and R⁵ are deuterium.

5. The compound of claim 1, wherein at least three of R¹, R², R³, R⁴, and R⁵ are deuterium.

6. The compound of claim 1, wherein R², R³, and R⁴ are deuterium; and R¹ and R⁵ are each hydrogen.

7. The compound of claim 1, wherein at least four of R¹, R², R³, R⁴, and R⁵ are deuterium.

8. A compound having the structural formula: 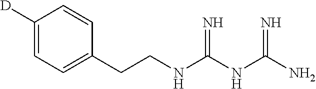 [SMILES: [2H]c1ccc(CCNC(=N)NC(=N)N)cc1]
or a pharmaceutically acceptable salt, solvate, or stereoisomer thereof.

9. The compound of claim 8, wherein the position represented as D has deuterium enrichment of at least 98%.

10. The compound of claim 8, wherein the position represented as D has deuterium enrichment of at least 90%.

11. The compound of claim 8, wherein the position represented as D has deuterium enrichment of at least 50%.

12. The compound of claim 8, wherein the position represented as D has deuterium enrichment of at least 10%.

13. The compound of claim 1 wherein the compound has a structural formula selected from the group consisting of: 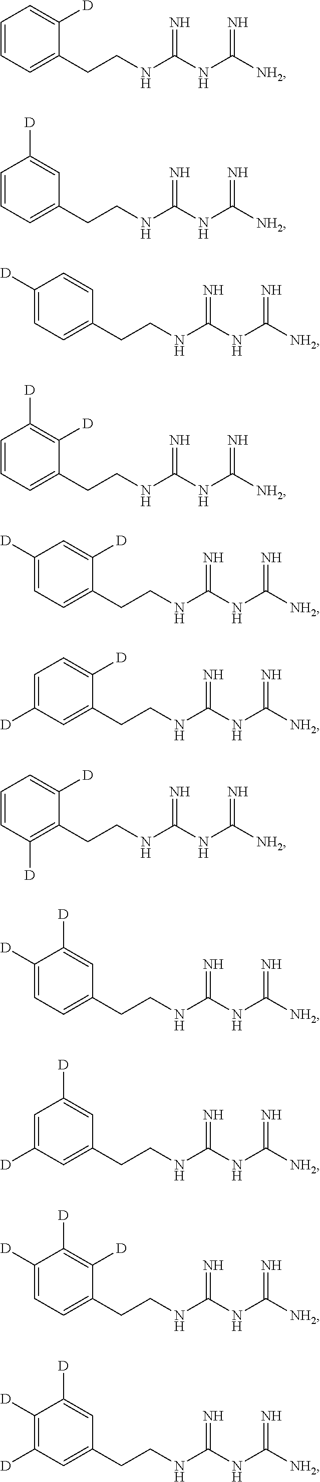 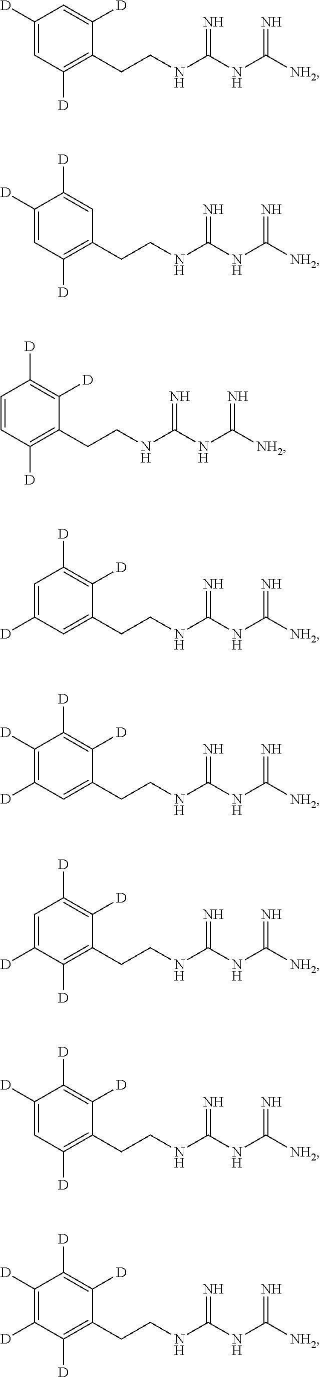
and a pharmaceutically acceptable salt, solvate, or stereoisomer thereof.

14. A pharmaceutical composition comprising a compound of any one claims 1-13 and a pharmaceutically acceptable carrier thereof.

15. A method of treating cancer in a subject, comprising administering to a subject a therapeutically effective amount of a compound of Formula (I) or a pharmaceutically acceptable salt, solvate, or stereoisomer thereof: 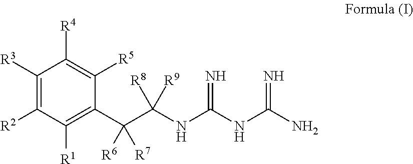
wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.

16. The method of claim 15, wherein the compound of Formula (I) has the structure: 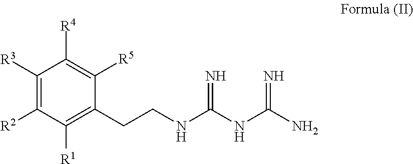
wherein
each of R₁, R², R³, R⁴, and R⁵ is hydrogen or deuterium; wherein at least one R¹, R², R³, R⁴, or R⁵ is deuterium.

17. The method of claim 16, wherein the compound of Formula (II) has the structural formula:

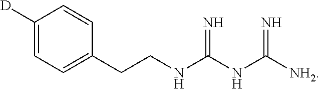 [SMILES: [2H]c1ccc(CCNC(=N)NC(=N)N)cc1]

18. The method of claim 15, wherein the cancer is selected from IDH1 mutant cancers and cancers with LKB1 deficient tumors.

19. A method of treating diabetes in a subject, comprising administering to a subject a therapeutically effective amount of a compound of Formula (I) or a pharmaceutically acceptable salt, solvate, or stereoisomer thereof: 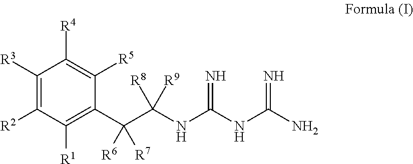
wherein
each of R¹, R², R³, R⁴, R⁵, R⁶, R⁷, R⁸, and R⁹ is hydrogen or deuterium, provided that at least one of R¹, R², R³, R⁴, R⁵, R⁶, R⁷, R⁸, or R⁹ is deuterium.
